(19) United States
(12) Patent Application Publication
(10) Pub. No.: US 2021/0228739 A1
(43) Pub. Date: Jul. 29, 2021

(54) RAAV VECTORS ENCODING OF LYSOSOMAL BETA-GALACTOSIDASE (GLB1) AND CATHEPSIN A

(71) Applicant: University of Massachusett, Boston, MA (US)

(72) Inventor: Miguel Sena Esteves, Westford, MA (US)

(73) Assignee: University of Massachusetts, Boston, MA (US)

(21) Appl. No.: 17/054,791

(22) PCT Filed: May 15, 2019
(86) PCT No.: PCT/US19/32365

§ 371 Date: Nov. 12, 2020

Related U.S. Application Data
(60) Provisional application No. 62/672,004, filed on May 15, 2018.

(51) Int. Cl. A61K 48/00; C12N 9/38; C12N 9/64; C12N 15/86

(52) U.S. Cl. CPC A61K 48/005; C12N 9/2471; C12N 2800/40; C12N 15/86; C12N 2750/14143; C12N 9/6424

(57) ABSTRACT

Aspects of the disclosure relate to methods for co-administration of recombinant AAVs (rAAVs). In some embodiments, the methods comprise administering one or more rAAVs engineered to express a transgene encoding β-galactosidase (GLB1), Cathepsin A (CTSA), and/or β-galactosidase (GLB1) and Cathepsin A (CTSA). In some embodiments, the disclosure provides methods for treating lysosomal storage disorders, such as GM-1 gangliosidosis, using compositions described by the disclosure.

### RELATED APPLICATIONS

This Application is a national stage filing under 35 U.S.C. § 371 of international PCT application, PCT/US2019/032365, filed May 15, 2019, which claims priority under 35 U.S.C. § 119(e) to U.S. provisional patent application, U.S. Ser. No. 62/672,004, filed May 15, 2018, the entire contents of each of which are which is incorporated herein by reference.

### BACKGROUND OF INVENTION

GM-1 gangliosidosis is an autosomal recessive lysosomal storage disease (LSD) caused by deficiency in β-galactosidase, resulting from mutations in the GLB1 gene. β-galactosidase deficiency causes decreased breakdown and increased build-up of gangliosides in lysosomes of the central nervous system (CNS). Cathepsin A (CTSA, also referred to as protective protein/cathepsin A, “PPCA”) is a carboxypeptidase enzyme which also appears to act as a protective protein in the lysosome.

### SUMMARY OF INVENTION

Aspects of the disclosure relate to recombinant AAV vectors for gene delivery. The disclosure is based, in part, on compositions (e.g., rAAV vectors and rAAVs) and methods for co-expression of β-galactosidase and Cathepsin (e.g., Cathepsin A) in a subject, which results in increased β-galactosidase expression and activity in serum and liver of the subject. In some embodiments, co-expression of β-galactosidase and Cathepsin (e.g., Cathepsin A) is an effective therapy for GM-1 gangliosidosis, a condition caused by decreased breakdown of gangliosides by β-galactosidase in the central nervous system (CNS).

Accordingly, in some aspects, the disclosure provides a method comprising administering to a cell: a first isolated nucleic acid comprising a transgene engineered to express a β-galactosidase (GLB) protein; and a second isolated nucleic acid comprising a transgene engineered to express a cathepsin protein.

In some embodiments, a cell is in vivo. In some embodiments, a cell is in a subject. In some embodiments, a subject is a human.

In some aspects, the disclosure provides a method for treating a lysosomal storage disease, the method comprising administering to a subject having a lysosomal storage disease: a first isolated nucleic acid comprising a transgene engineered to express a β-galactosidase (GLB) protein; and a second isolated nucleic acid comprising a transgene engineered to express a cathepsin protein.

In some aspects, the disclosure provides a method for treating a lysosomal storage disease, the method comprising administering to a subject having a lysosomal storage disease a composition or an rAAV as described by the disclosure. In some embodiments, a lysosomal storage disease is GM1-gangliosidosis.

In some embodiments, GLB protein is a human GLB protein (e.g., hGLB1) or a mouse GLB protein (e.g., mGLB1). In some embodiments, a GLB protein comprises or consists of the sequence set forth in SEQ ID NO: 5 or 6.

In some embodiments, a cathepsin protein is a cathepsin A protein (CTSA). In some embodiments, a cathepsin A protein is human cathepsin A protein (hCTSA) or mouse cathepsin A protein (mCTSA). In some embodiments, a CTSA protein comprises or consists of the sequence set forth in SEQ ID NO: 7 or 8.

In some embodiments, a first isolated nucleic acid comprises adeno-associated virus (AAV) inverted terminal repeats (ITRs) flanking a transgene. In some embodiments, a second isolated nucleic acid comprises adeno-associated virus (AAV) inverted terminal repeats (ITRs) flanking a transgene. In some embodiments, at least one of the AAV ITRs flanking a transgene (e.g., a transgene of a first isolated nucleic acid and/or a transgene of a second isolated nucleic acid) is a mutant ITR (mTR) or a ΔITR.

In some embodiments, a transgene of a first isolated nucleic acid and/or a transgene of a second isolated nucleic acid further comprises a promoter. In some embodiments, a promoter is a CAG promoter.

In some embodiments, a first isolated nucleic acid is located on a plasmid. In some embodiments, a second isolated nucleic acid is located on a plasmid. In some embodiments, a first isolated nucleic acid and a second isolated nucleic acid are located on the same plasmid.

In some embodiments, a first isolated nucleic acid is encapsidated by an AAV capsid protein. In some embodiments, a second isolated nucleic acid is encapsidated by an AAV capsid protein. In some embodiments, a capsid protein is an AAV9 capsid protein.

In some embodiments, a first isolated nucleic acid and a second isolated nucleic acid are encapsidated together in the same rAAV. In some embodiments, the rAAV comprises an AAV9 capsid protein.

In some embodiments, a first isolated nucleic acid and/or a second isolated nucleic acid are administered by injection.

In some embodiments, a first isolated nucleic acid and a second isolated nucleic acid are administered separately. In some embodiments, a first isolated nucleic acid and a second isolated nucleic acid are administered together (e.g., as part of the same composition). In some embodiments, a first isolated nucleic acid and a second isolated nucleic acid are administered simultaneously (e.g., at the same time).

In some aspects, the disclosure provides an isolated nucleic acid comprising the sequence set forth in any one of SEQ ID NOs: 1-4, and 9-12.

In some aspects, the disclosure provides a composition comprising: a first recombinant adeno-associated virus (rAAV) comprising a transgene engineered to express a β-galactosidase (GLB) protein; and a second rAAV, comprising a transgene engineered to express a cathepsin protein. In some embodiments, a composition further comprises a pharmaceutically acceptable excipient.

In some aspects, the disclosure provides a recombinant adeno-associated virus (rAAV) comprising a transgene engineered to express: a β-galactosidase (GLB) protein; and a cathepsin protein.

In some embodiments, an rAAV comprises an AAV9 capsid protein. In some embodiments, the first rAAV and/or the second rAAV of a composition comprises an AAV9 capsid protein.

In some embodiments, a β-galactosidase protein is a human GLB protein (e.g., hGLB1) or a mouse GLB protein (e.g., mGLB1). In some embodiments, a GLB protein comprises or consists of the sequence set forth in SEQ ID NO: 5 or 6

In some embodiments, a cathepsin protein is a cathepsin A protein (CTSA). In some embodiments, a cathepsin A protein is human cathepsin A protein (hCTSA) or mouse cathepsin protein A (mCTSA). In some embodiments, a CTSA protein comprises or consists of the sequence set forth in SEQ ID NO: 7 or 8.

In some embodiments, a composition comprises a ratio of a first rAAV to a second rAAV selected from 0.5:1, 1:1, 1:2, 1:4, 1:10, 1:0.5, 2:1, 4:1, and 10:1. In some embodiments, a composition comprises a ratio of a second rAAV to a first rAAV selected from 0.5:1, 1:1, 1:2, 1:4, 1:10, 1:0.5, 2:1, 4:1, and 10:1.

In some embodiments, a composition is formulated for injection.

### BRIEF DESCRIPTION OF DRAWINGS

FIG. 1 shows an increase in lysosomal acid β-galactosidase activity measured in liver after gene transfer of mouse GLB1 (mGLB1) (β-galactosidase) or human GLB1 (hGLB1). The treatment groups were: (1) hGLB1, (2) PBS (control), and (3) mGLB1. rAAV engineered to express hGLB1 and mGLB1 were packaged in an AAV9 capsid protein.

FIGS. 2A-2C shows data related to IV injections of matched-dose AAV vectors encoding TdTomato and GFP in a shared backbone and co-transduction of the same cells in liver. FIG. 2A shows expression of AAV9-CB-GFP in liver at 20× magnification. FIG. 2B shows expression of AAV9-CB-TdTomato in liver at 20× magnification. FIG. 2C shows co-expression of AAV9-CB-TdTomato+AAV9-CB-GFP in liver at 20× magnification.

FIG. 3 shows co-delivery of hGLB1 and PPCA (Cathepsin A, CTSA) enhances β-galactosidase activity measured in liver. The treatment groups were: (1) mouse PPCA (mCTSA), (2) human PPCA (hCTSA), (3) human GLB1, (4) human GLB1+ mouse PPCA, (5) human GLB1+ human PPCA, and (6) PBS (control).

FIG. 4 shows fold increase in β-galactosidase activity in liver following co-delivery of hGLB1 and PPCA over PBS (control). The treatment groups were: (1) mouse PPCA, (2) human PPCA, (3) human GLB1, (4) human GLB1+ mouse PPCA, and (5) human GLB1+ human PPCA.

FIG. 5 shows co-delivery of hGLB1 and PPCA enhances β-galactosidase activity measured in serum. The treatment groups were: (1) mouse PPCA, (2) human PPCA, (3) human GLB1, (4) human GLB1+ mouse PPCA, (5) human GLB1+ human PPCA, and (6) PBS (control).

FIG. 6 shows the fold increase β-galactosidase activity in serum following co-delivery of hGLB1 and PPCA over PBS (control). The treatment groups were: (1) mouse PPCA, (2) human PPCA, (3) human GLB1, (4) human GLB1+ mouse PPCA, and (5) human GLB1+ human PPCA.

FIGS. 7A and 7B shows increased expression of mature β-galactosidase expression in liver by co-administration of AAV9 vectors encoding GLB1 and PPCA proteins. The treatment groups were: (1) PBS (control), (2) mouse PPCA, (3) human PPCA, (4) human GLB1, (5) human GLB1+ mouse PPCA, and (6) human GLB1+ human PPCA. FIG. 7A is a western blot of human β-galactosidase expression. FIG. 7B is a β-galactosidase activity assay from the liver samples blotted for expression in FIG. 7A.

FIG. 8 shows a recombinant AAV (rAAV) vector comprising a transgene encoding hGLB1 under control of a CAG promoter.

FIG. 9 shows an rAAV vector comprising a transgene encoding mGLB1 under control of a CAG promoter.

FIG. 10 shows an rAAV vector comprising a transgene encoding hCTSA under control of a CAG promoter.

FIG. 11 shows an rAAV vector comprising a transgene encoding mCTSA under control of a CAG promoter.

### DETAILED DESCRIPTION OF INVENTION

In some aspects, the disclosure relates to compositions and methods useful in the treatment of lysosomal storage disorders, for example GM-1 gangliosidosis. The disclosure is based, in part, on recombinant AAV vectors (e.g., isolated nucleic acids) and recombinant adeno-associated viruses (rAAVs) comprising expression cassettes configured for expression of β-galactosidase (GLB) and/or Cathepsin (e.g., Cathepsin A, CTSA). In some embodiments, co-expression or co-administration (e.g., simultaneous expression or simultaneous administration) of isolated nucleic acids, rAAVs and/or compositions described by the disclosure results in stabilization of GLB protein by Cathepsin, and thus improved expression of mature GLB protein in a cell (e.g., cell of a subject).

### Isolated Nucleic Acids

In some aspects, the disclosure provides isolated nucleic acids (e.g., expression constructs, such as rAAV vectors) that are useful in expressing therapeutic transgenes (e.g., GLB1, CTSA, or a combination thereof) in the cells of a subject.

As used herein, “a β-galactosidase protein” refers to an enzyme that hydrolyzes terminal non-reducing β-D galactose residues in β-D-galactosides. In humans, β-galactosidase is encoded by the GLB1 gene (Gene ID: 2720), which is conserved in chimpanzee, Rhesus monkey, dog, cow, mouse, rat, chicken, zebrafish, mosquito, C. elegans, A. thaliana, rice, and frog. Generally, β-galactosidase proteins comprise a conserved glycosyl hydrolase family 35 domain active domain at the N terminus, as well as unstructured C-terminal regions.

In some embodiments, a β-galactosidase protein is encoded by a human GLB1 gene, which comprises the sequence set forth in NCBI Ref. Seq ID No: NG_009005.1. In some embodiments, a β-galactosidase protein is encoded by a mouse GLB1 gene, which comprises the sequence set forth in NCBI Ref. Seq ID No: NM_009752.2. In some embodiments, a β-galactosidase protein comprises an amino acid sequence that is 99% identical, 95% identical, 90% identical, 80% identical, 70% identical, 60% identical, or 50% identical to the amino acid sequence encoded by the nucleic acid sequence set forth in either Ref Seq ID No: NG_009005.1 or NM_009752.2. In some embodiments, a β-galactosidase protein is encoded by the nucleic acid sequence set forth in SEQ ID NO: 1 or 2. In some embodiments, a β-galactosidase protein is encoded by a nucleic acid sequence that is 99% identical, 95% identical, 90% identical, 80% identical, 70% identical, 60% identical, or 50% identical to either SEQ ID NO: 1 or 2.

In some embodiments, a β-galactosidase protein is comprises or consists of the amino acid sequence set forth in SEQ ID NO: 5 or 6. In some embodiments, a β-galactosidase protein is 99% identical, 95% identical, 90% identical, 80% identical, 70% identical, or 50% identical to SEQ ID NO: 5 or 6. In some embodiments, a β-galactosidase protein is a fragment (e.g., portion) of a GLB protein (e.g., a fragment or portion of a GLB protein that retains the catalytic function of wild-type GLB protein). In some embodiments, a fragment (or portion) of a GLB protein comprises or consists of an amino acid sequence that has 99%, 98%, 95%, 90%, 80%, 70%, 60% or 50% of the amino acids set forth in in SEQ ID NO: 5 or 6. In some embodiments, a fragment (or portion) of a GLB protein comprises or consists of an amino acid sequence that is truncated (e.g., shortened) by about 1, 2, 3, 4, 5, 10, 20, 30, 40, 50, 60, 70, 80, 90, 100, 200, or 500 amino acids relative to the amino acid sequence set forth in in SEQ ID NO: 5 or 6.

As used herein, a “cathepsin protein” refers to one of a family of protease enzymes that are generally active at low pH and are found almost entirely within lysosomes. Examples of cathepsin proteins include but are not limited to Cathepsin A, Cathepsin B, Cathepsin C, Cathepsin D, Cathepsin E, Cathepsin F, Cathepsin G, Cathepsin H, Cathepsin K, Cathepsin L1, Cathepsin L2 or V, Cathepsin O, Cathepsin S, Cathepsin W, and Cathepsin X or Z.

In some embodiments, a cathepsin protein is Cathepsin A (also known as PPCA), which is encoded by the CTSA gene (Gene ID: 5476). In some embodiments, cathepsin proteins are conserved in chimpanzee, Rheusus monkey, dog, cow, mouse, rat, chicken, zebrafish, C. elegans, S. cerevisiae, K. lactis, E. gossypii, S. pombe, M. oryzae, N. crassa, A. thaliana, rice, and frog. Cathepsin proteins encoded by these homologous genes typically include a carboxypeptidase active domain, as well as unstructured regions.

In some embodiments, a Cathepsin A (CTSA) protein is encoded by a human CTSA (hCTSA) gene, which comprises the sequence set forth in NCBI Ref. Seq ID No: NG_008921.1. In some embodiments, a Cathepsin A (CTSA) protein is encoded by a mouse CTSA (mCTSA) gene, which comprises the sequence set forth in NCBI Ref. Seq ID No: NM_008906.4. In some embodiments, a cathepsin protein (e.g., CTSA) comprises an amino acid sequence that is 99% identical, 95% identical, 90% identical, 80% identical, 70% identical, 60% identical, or 50% identical to an amino acid sequence encoded by the nucleic acid sequence set forth in either Ref Seq ID No: NG_008921.1 or NM_008906.4. In some embodiments, a cathepsin protein is encoded by the nucleic acid sequence set forth in SEQ ID NO: 3 or 4. In some embodiments, a cathepsin protein is encoded by a nucleic acid sequence that is 99% identical, 95% identical, 90% identical, 80% identical, 70% identical, 60% identical, or 50% identical to either SEQ ID NO: 3 or 4.

In some embodiments, a cathepsin protein (e.g., CTSA) is comprises or consists of the amino acid sequence set forth in SEQ ID NO: 7 or 8. In some embodiments, a cathepsin (e.g., CTSA) protein is 99% identical, 95% identical, 90% identical, 80% identical, 70% identical, or 50% identical to SEQ ID NO: 7 or 8. In some embodiments, a cathepsin protein is a fragment (e.g., portion) of a cathepsin protein (e.g., a fragment or portion of a cathepsin protein that retains the proteolytic function of wild-type CTSA protein). In some embodiments, a fragment (or portion) of a cathepsin protein comprises or consists of an amino acid sequence that has 99%, 98%, 95%, 90%, 80%, 70%, 60% or 50% of the amino acids set forth in in SEQ ID NO: 7 or 8. In some embodiments, a fragment (or portion) of a cathepsin protein comprises or consists of an amino acid sequence that is truncated (e.g., shortened) by about 1, 2, 3, 4, 5, 10, 20, 30, 40, 50, 60, 70, 80, 90, 100, 200, or 500 amino acids relative to the amino acid sequence set forth in in SEQ ID NO: 7 or 8.

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A signal of a transgene. For example, the region may be positioned between the first base of the poly-A signal and 2000 nucleotides upstream of the first base. The region may be positioned between the first base of the poly-A signal and 1000 nucleotides upstream of the first base. The region may be positioned between the first base of the poly-A signal and 500 nucleotides upstream of the first base. The region may be positioned between the first base of the poly-A signal and 250 nucleotides upstream of the first base. The region may be positioned between the first base of the poly-A signal and 150 nucleotides upstream of the first base. The region may be positioned between the first base of the poly-A signal and 100 nucleotides upstream of the first base. The region may be positioned between the first base of the poly-A signal and 50 nucleotides upstream of the first base. The region may be positioned between the first base of the poly-A signal and 20 nucleotides upstream of the first base. In some embodiments, the region is positioned between the last nucleotide base of a promoter sequence and the first nucleotide base of a poly-A signal sequence.

In some cases, the region may be positioned downstream of the last base of the poly-A signal of a transgene. The region may be between the last base of the poly-A signal and a position 2000 nucleotides downstream of the last base. The region may be between the last base of the poly-A signal and a position 1000 nucleotides downstream of the last base. The region may be between the last base of the poly-A signal and a position 500 nucleotides downstream of the last base. The region may be between the last base of the poly-A signal and a position 250 nucleotides downstream of the last base. The region may be between the last base of the poly-A signal and a position 150 nucleotides downstream of the last base.

It should be appreciated that in cases where a transgene encodes more than one polypeptide, each polypeptide may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is a P2 promoter. In some embodiments, a promoter is a chicken β-actin (CBA) promoter. In some embodiments, a promoter is two CBA promoters. In some embodiments, a promoter is two CBA promoters separated by a CMV enhancer. In some embodiments, a promoter is a CAG promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding a protein it may be desirable to drive expression of the first protein coding region using a first promoter sequence (e.g., a first promoter sequence operably linked to the protein coding region), and to drive expression of the second protein coding region with a second promoter sequence (e.g., a second promoter sequence operably linked to the second protein coding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (pol III) promoter sequence. Non-limiting examples of pol III promoter sequences include U6 and H1 promoter sequences. In some embodiments, the second promoter sequence (e.g., the promoter sequence driving expression of the second protein) is a RNA polymerase II (pol II) promoter sequence. Non-limiting examples of pol II promoter sequences include T7, T3, SP6, RSV, and cytomegalovirus promoter sequences. In some embodiments, a pol III promoter sequence drives expression of the first protein coding region. In some embodiments, a pol II promoter sequence drives expression of the second protein coding region.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the therapeutic protein is useful for treatment or prevention of lysosomal storage diseases such as GM-1 gangliosidosis, including, but not limited to, β-ganglioside and Cathepsin A.

In some embodiments, the isolated nucleic acid or rAAV vector disclosed also comprises a miRNA binding site. MicroRNAs (miRNAs) are small nucleic acids which appear to play a role in regulating a broad range of cellular processes, and changes in miRNA expression have been implicated in human disease. As used herein, the term “miRNA binding site,” with reference to a miRNA inhibitor, refers to a sequence of nucleotides in a miRNA inhibitor that are sufficiently complementary with a sequence of nucleotides in a miRNA to effect base pairing between the miRNA inhibitor and the miRNA. Typically, a miRNA binding site comprises a sequence of nucleotides that are sufficiently complementary with a sequence of nucleotides in a miRNA to effect base pairing between the miRNA inhibitor and to thereby inhibit binding of the miRNA to a target mRNA. The typical miRNA inhibitor of the invention is a nucleic acid molecule that comprises at least one miRNA binding site, e.g., an miR-122 binding site. The miRNA inhibitors may comprise 1 miRNA binding site, 2 miRNA binding sites, 3 miRNA binding sites, 4 miRNA binding sites, 5 miRNA binding sites, 6 miRNA binding sites, 7 miRNA binding sites, 8 miRNA binding sites, 9 miRNA binding sites, 10 miRNA binding sites, or more miRNA binding sites. MiRNAs are expressed endogenously throughout the body in a tissue-specific manner (i.e. miR-122 is highly expressed in the liver). In some embodiments, the miRNA binding site(s) incorporated into the isolated nucleic acid or rAAV vector disclosed represses expression of the transgene in an off-target tissue.

### Bicistronic Nucleic Acid Constructs

Some aspects of this invention provide bicistronic nucleic acid constructs. The term “cistron” refers to a nucleic acid cassette sufficient for expression of a gene product. In some embodiments, a cistron is an expression cassette. Accordingly, some aspects of this invention provide nucleic acid constructs comprising two or more cistrons, for example, two or more expression cassettes. The term “expression cassette” refers to a nucleic construct comprising nucleic acid elements sufficient for the expression of a gene product. Typically, an expression cassette comprises a nucleic acid encoding a gene product operatively linked to a promoter sequence. Encoding sequences can be operatively linked to regulatory sequences in sense or antisense orientation. In some embodiments, the promoter is a heterologous promoter. The term “heterologous promoter”, as used herein, refers to a promoter that does is not found to be operatively linked to a given encoding sequence in nature. In some embodiments, an expression cassette may comprise additional elements, for example, an intron, an enhancer, a polyadenylation site, a woodchuck hepatitis virus post-transcriptional regulatory element (WPRE), and/or other elements known to affect expression levels of the encoding sequence. Without wishing to be bound by theory, inclusion of an intron in an expression cassette, for example, between the transcriptional start site and an encoding nucleic acid sequence, for example, a protein-encoding cDNA sequence, is believed to result in increased expression levels of the encoding nucleic acid and the encoded gene product as compared to an expression construct not including an intron.

The term “intron” refers to a nucleic acid sequence in an expression cassette that is removed after transcription of a primary transcript by a cellular process termed splicing. Intron sequences generally comprise a splice donor and a splice acceptor and sequences of such donor and acceptor sites are well known to those of skill in the art. “Chimeric intron” as used herein, are composed of nucleic acid sequences from two or more different sources.

Some aspects of this invention provide bicistronic expression constructs comprising two or more expression cassettes in various configurations.

In different embodiments, bicistronic expression constructs are provided in which the expression cassettes are positioned in different ways. For example, in some embodiments, a multicistronic expression construct is provided in which a first expression cassette is positioned adjacent to a second expression cassette. In some embodiments, the first expression cassette and the second expression cassette are operably linked by a bidirectional promoter, wherein the first expression cassette and the second expression cassette are flanked by adeno-associated virus (AAV) inverted terminal repeats (ITRs).

In different embodiments, bicistronic expression constructs are provided in which the expression cassettes are oriented in different ways. For example, in some embodiments, bicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g., 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1-promoter2/encoding sequence 2-3′,

the expression cassettes are in the same orientation, the arrows indicate the direction of transcription of each of the cassettes. For another example, if a given nucleic acid construct comprises a sense strand comprising two expression cassettes in the configuration

5′-promoter 1/encoding sequence 1-encoding sequence 2/promoter 2-3′,

the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR or a second expression cassette (e.g., transcription of the expression cassette proceeds in the same direction as transcription of the AAV ITR or second expression cassette), or in opposite orientation (e.g., transcription of the expression cassette proceeds in the opposite direction (e.g., distally) as transcription of the AAV ITR or second expression cassette). AAV ITRs are directional. For example, an AAV construct comprising a 5′ITR would be in the same orientation as the GLB1 expression cassette. In another example, an AAV construct comprising the 5′ITR would be in the opposite orientation as the GLB1 expression cassette.

A large body of evidence suggests that bicistronic expression constructs often do not achieve optimal expression levels as compared to expression systems containing only one cistron. One of the suggested causes of sub-par expression levels achieved with bicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). Various strategies have been suggested to overcome the problem of promoter interference, for example, by producing bicistronic expression constructs comprising only one promoter driving transcription of multiple encoding nucleic acid sequences separated by internal ribosomal entry sites, or by separating cistrons comprising their own promoter with transcriptional insulator elements. All suggested strategies to overcome promoter interference are burdened with their own set of problems, though. For example, single-promoter driven expression of multiple cistrons usually results in uneven expression levels of the cistrons. Further some promoters cannot efficiently be isolated and isolation elements are not compatible with some gene transfer vectors, for example, some retroviral vectors.

In some embodiments of this invention, a bicistronic expression construct is provided that allows efficient expression of a first encoding nucleic acid sequence driven by a first promoter and of a second encoding nucleic acid sequence driven by a second promoter without the use of transcriptional insulator elements. Various configurations of such bicistronic expression constructs are provided herein, for example, expression constructs harboring a first expression cassette comprising an intron and a second expression cassette comprising an intron, wherein the first expression cassette and second expression cassette are under the control of separate promoters located proximal to the AAV ITRs that flank the first expression cassette and the second expression cassette. In some embodiments, the first expression cassette and the second expression cassette are operably linked by a bidirectional promoter and are flanked by AAV ITRs.

In some embodiments, bicistronic expression constructs are provided allowing for efficient expression of two or more encoding nucleic acid sequences. In some embodiments, the bicistronic expression construct comprises two expression cassettes. In some embodiments, a first expression cassette of a bicistronic expression construct as provided herein comprises an RNA polymerase II promoter and a second expression cassette comprises an RNA polymerase III promoter. In some embodiments, a first expression cassette comprises a P2 promoter and a second expression cassette comprises a P2 promoter. In some embodiments, a first expression cassette and a second expression cassette are operably linked by a bidirectional promoter. In some embodiments, the bicistronic expression construct provided is a recombinant AAV (rAAV) construct.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, and AAV.PHP.B. In some embodiments, an AAV capsid protein is of a serotype derived from a non-human primate, for example AAVrh8 serotype. In some embodiments, an AAV capsid protein is of a serotype derived for broad and efficient CNS transduction, for example AAV.PHP.B. In some embodiments, the capsid protein is of AAV serotype 9.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a protein (e.g., β-galactosidase and Cathepsin A proteins). In some embodiments, the host cell is a mammalian cell, a yeast cell, a bacterial cell, an insect cell, a plant cell, or a fungal cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product from a transcribed gene. The foregoing methods for packaging recombinant vectors in desired AAV capsids to produce the rAAVs of the disclosure are not meant to be limiting and other suitable methods will be apparent to the skilled artisan.

### Methods for Treating Lysosomal Storage Diseases

Methods for delivering a transgene to a subject are provided by the disclosure. The methods typically involve administering to a cell or a subject an effective amount of isolated nucleic acid encoding β-galactosidase (GLB1) and CTSA (Cathepsin A) (e.g., wild-type GLB1 and/or CTSA, codon-optimized GLB1 and/or CTSA, or any combination of the foregoing) proteins. β-galactosidase is the lysosomal protein/enzyme in human that breaks down GM-1 ganglioside. As such, mutations which decrease β-galactosidase activity generally result in the toxic accumulation of GM-1 ganglioside and a lysosomal storage disorder. Human lysosomal β-galactosidase exists in a complex with the lysosomal protective protein Cathepsin A and the enzyme neuraminidase. Thus, in some embodiments, compositions described by the disclosure are useful for treating lysosomal storage diseases, such as GM1-gangliosidosis.

As used herein, a “lysosomal storage disorder” refers to an inherited metabolic disease characterized by abnormal build-up of biomolecules in cells, particularly neurons, resulting from bi-allelic mutations in enzymes which catalyze breakdown of the biomolecules. In preferred embodiments, a lysosomal storage disorder is GM-1 gangliosidosis, resulting from biallelic mutations in GLB1 gene, resulting in reduced or loss of function of the β-galactosidase protein of the subject. “Biallelic mutations” refers to both copies of a gene, in this case GLB1, CTSA, or NEU1, possess alterations in amino acid sequence. The progressive build-up of GM-1 gangliosides in lysosomes leads to the destruction of neurons. In some embodiments, a lysosomal storage disorder is sialidosis, resulting from biallelic mutations in the NEU1 gene, resulting in reduced or loss of function of the neuraminidase protein of the subject. In some embodiments, a lysosomal storage disorder is galactosialidosis, resulting from biallelic mutations in the CTSA gene, resulting in reduced or loss of function of the Cathepsin A protein of the subject.

A cell is typically a mammalian cell. In some embodiments, a cell is in a subject (e.g., in vivo). In some embodiments, a subject is a mammalian subject, for example a human. In some embodiments, a cell is a nervous system cell (central nervous system cell or peripheral nervous system cell), for example a neurons (e.g., unipolar neurons, bipolar neurons, Basket cells, Betz cells, Lugaro cells, spiny neurons, Purkinje cells, Pyrimidal cells, Renshaw cells, Granule cells, motor neurons, spindle cells, etc.) or glial cells (e.g., astrocytes, oligodendrocytes, ependymal cells, radial glia, Schwann cells, Satellite cells, etc.).

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to compensate for reduction or loss of function of a protein resulting from mutation of a gene (e.g., GLB1 or CTSA), to extend the lifespan of a subject, to improve in the subject one or more symptoms of disease (e.g., a symptom of GM-1 gangliosidosis), etc. The effective amount will depend on a variety of factors such as, for example, the species, age, weight, health of the subject, and the tissue to be targeted, and may thus vary among subject and tissue as described elsewhere in the disclosure.

In some aspects, the disclosure relates to the discovery that co-administration of an isolated nucleic acid encoding GLB1 and an isolated nucleic acid encoding CTSA to a cell results, in some embodiments, in stabilization of the GLB1 protein by CTSA and increased production of mature GLB1 in the cell. In some embodiments, administration of one or more isolated nucleic acids, rAAVs, or compositions as described herein results in improved (e.g., increased) expression or activity of a transgene (e.g., β-galactosidase) in a cell or a subject.

In some embodiments, “improved” or “increased” expression or activity of a transgene is measured relative to expression or activity of that transgene in a cell or subject who has not been administered one or more isolated nucleic acids, rAAVs, or compositions as described herein. For example, in some embodiments, “improved” or “increased” expression or activity of β-galactosidase in a cell or subject is measured relative to a cell or subject who has been administered a transgene encoding β-galactosidase in the absence of administration of a transgene encoding CTSA. In some embodiments, methods described by the disclosure result in β-galactosidase expression and/or activity in a subject that is increased between 2-fold and 100-fold (e.g., 2-fold, 5-fold, 10-fold, 50-fold, 100-fold, etc.) relative to the β-galactosidase expression and/or activity of a subject who has not been administered one or more compositions described by the disclosure.

### Modes of Administration

In some aspects, the disclosure relates to the discovery that co-expression or co-administration of a GLB protein and a cathepsin protein results in stabilization of the GLB protein by the cathepsin protein.

The timing and order of administration of a GLB protein and a cathepsin protein can vary. In some embodiments, an isolated nucleic acid or rAAV encoding a GLB protein and an isolated nucleic acid or rAAV encoding a cathepsin protein are administered separately (e.g., administered as separate compositions). In some embodiments, an isolated nucleic acid or rAAV encoding a GLB protein and an isolated nucleic acid or rAAV encoding a cathepsin protein are administered together (e.g., as part of the same composition or at the same time).

In some embodiments, an isolated nucleic acid or rAAV encoding a GLB protein and an isolated nucleic acid or rAAV encoding a cathepsin protein are administered simultaneously (e.g., at the same time). In some embodiments, an isolated nucleic acid or rAAV encoding a GLB protein and an isolated nucleic acid or rAAV encoding a cathepsin protein are administered within 10 seconds, 30 seconds, 1 minute, 2 minutes, 3 minutes, 4 minutes, 5 minutes, 10 minutes, 30 minutes, 1 hour, 2 hours, 8 hours, 12 hours, 24 hours, 2 days, 7 days, or more than 7 days of one another. In some embodiments, an isolated nucleic acid or rAAV encoding a GLB protein is administered 10 seconds, 30 seconds, 1 minute, 2 minutes, 3 minutes, 4 minutes, 5 minutes, 10 minutes, 30 minutes, 1 hour, 2 hours, 8 hours, 12 hours, 24 hours, 2 days, 7 days, or more than 7 days before an isolated nucleic acid or rAAV encoding a cathepsin protein. In some embodiments, an isolated nucleic acid or rAAV encoding a GLB protein is administered 10 seconds, 30 seconds, 1 minute, 2 minutes, 3 minutes, 4 minutes, 5 minutes, 10 minutes, 30 minutes, 1 hour, 2 hours, 8 hours, 12 hours, 24 hours, 2 days, 7 days, or more than 7 days after an isolated nucleic acid or rAAV encoding a cathepsin protein. In some embodiments, GLB protein and a cathepsin protein results in expression of the GLB protein and the cathepsin protein in the same cell (e.g., a CNS cell).

The isolated nucleic acids, rAAVs and compositions of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), thalamus, spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, an rAAV as described in the disclosure are administered by intravenous injection. In some embodiments, rAAVs are administered by intracerebral injection. In some embodiments, rAAVs are administered by intrathecal injection. In some embodiments, rAAVs are administered by intrastriatal injection. In some embodiments, rAAVs are delivered by intracranial injection. In some embodiments, rAAVs are delivered by cisterna magna injection. In some embodiments, the rAAV are delivered by cerebral lateral ventricle injection.

Aspects of the instant disclosure relate to compositions comprising a recombinant AAV comprising a capsid protein and a nucleic acid encoding a transgene, wherein the transgene comprises a nucleic acid sequence encoding one or more proteins. In some embodiments, each protein comprises a sequence set forth in any one of SEQ ID NO: 5 to 8. In some embodiments, the nucleic acid further comprises AAV ITRs. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is administered to the subject during a pre-symptomatic stage of the lysosomal storage disease. In some embodiments, the pre-symptomatic stage of the lysosomal storage disease occurs between birth (e.g., perinatal) and 4-weeks of age.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.) Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

### Example 1: Administration of β-Galactosidase and Cathepsin A in Mice

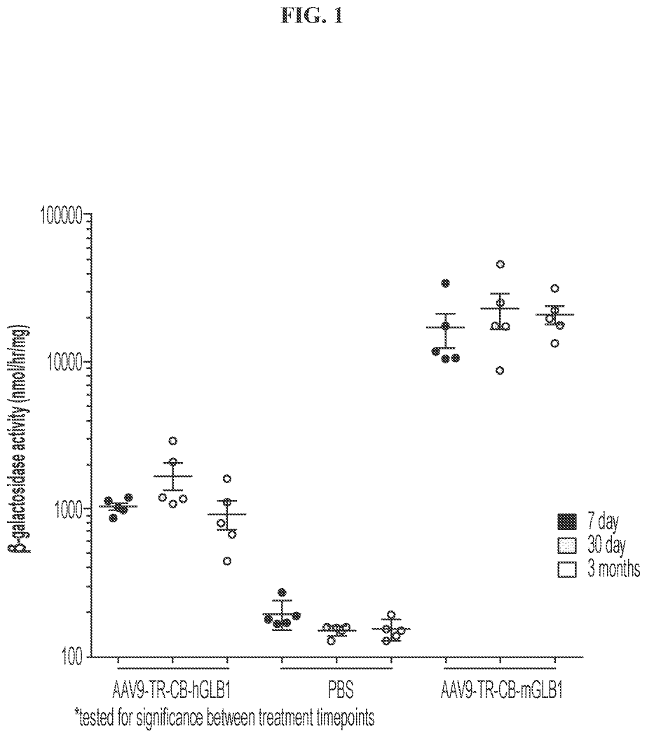

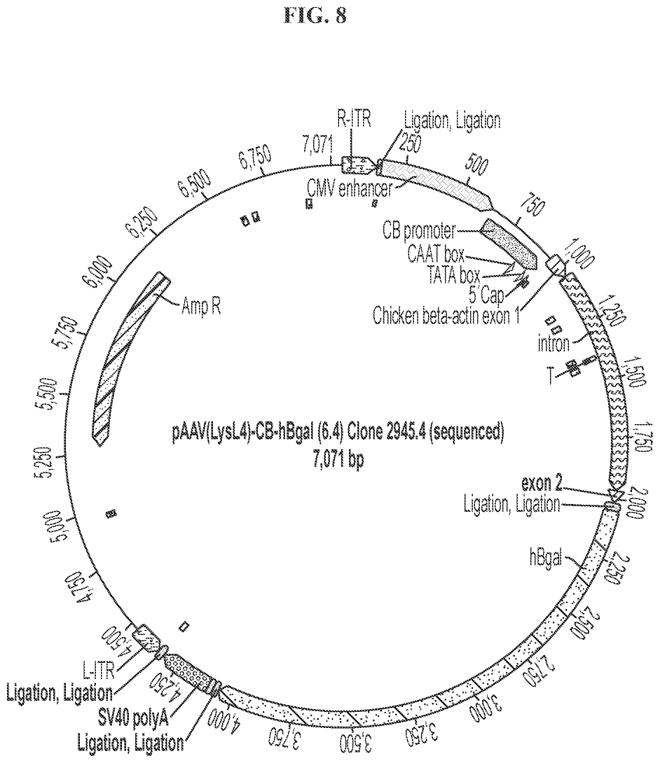

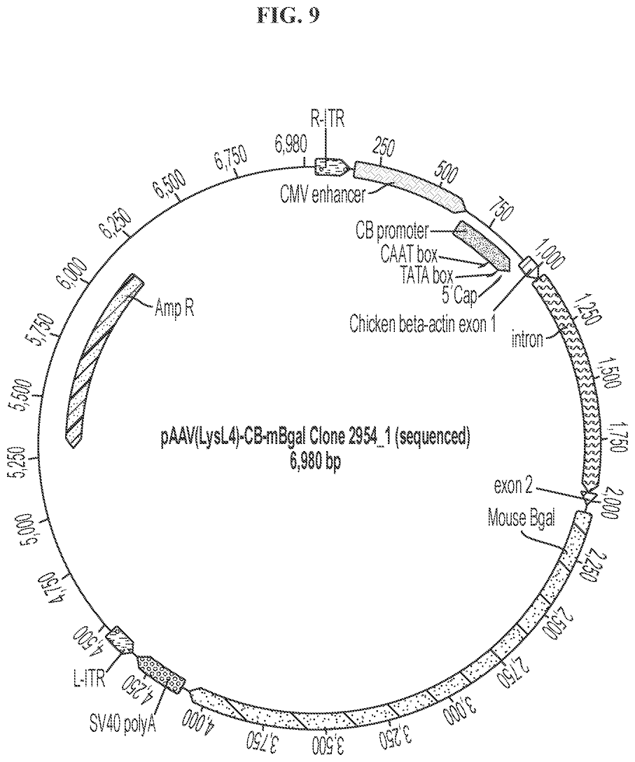

C57BL/6 mice were injected with PBS (control), AAV9-hGLB1 (human β-galactosidase) or AAV9-mGLB1 (mouse human β-galactosidase) under operative control of a CAG promoter (FIG. 8 and FIG. 9). Treated mice received 2.25×10¹¹ vg of each AAV9 vector. Each cohort (n=5) was euthanized at 7 days, 30 days, and 30 months post-treatment (FIG. 1). β-galactosidase enzyme activity was measured from liver samples using the fluorogenic substrate β-4-methylumbelliferone (4-MU), which fluoresces upon cleavage by β-galactosidase. Increases in β-galactosidase activity were observed in both treatment groups relative to PBS-injected control mice. Activity of mGLB1 was observed to be higher than hGLB1.

### Example 2: Co-Administration of β-Galactosidase and Cathepsin A in Mice

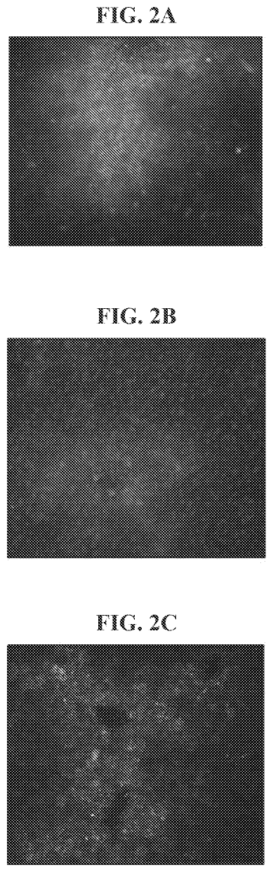

A test cohort of C57BL/6 mice were injected with AAV9 vectors encoding GFP or TdTomato protein to assess the degree of liver co-transduction (FIG. 2). Two mice from each cohort were injected intravenously with 1×10¹² vg/AAV in a total volume of 200 μL with AAV9-CB-GFP (FIG. 2A), AAV9-CB-TdTomato (FIG. 2B), or AAV9-CB-GFP and AAV9-CB-TdTomato (FIG. 2C).

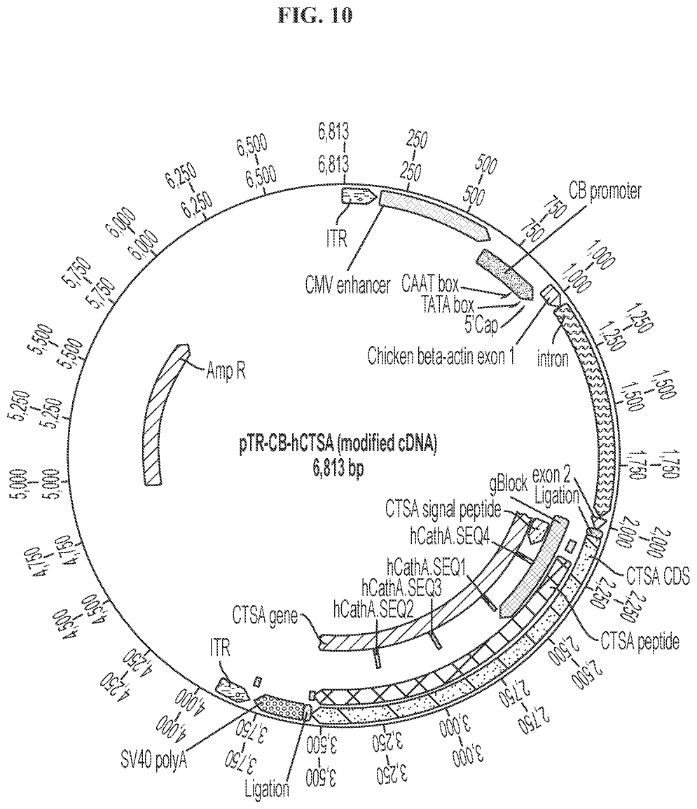

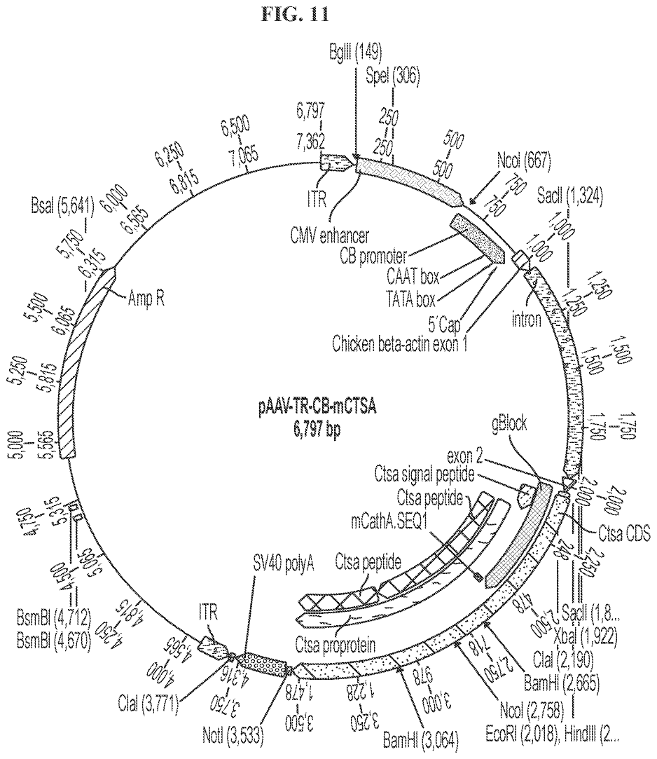

In another experiment, C57BL/6 mice were injected with recombinant AAV9 vectors encoding human GLB1 (β-galactosidase protein) and/or either human or mouse CTSA (Cathepsin A, also referred to as PPCA) genes operably linked to a CAG promoter (FIG. 10 and FIG. 11). Each mouse received 1×10¹² vg/AAV via the tail vein in a total of 250 μL. Each treatment group (human Cathepsin A, human β-galactosidase, human β-galactosidase+mouse Cathepsin A, human β-galactosidase+human Cathepsin A, or PBS control) contained 5 mice, except the mouse Cathepsin A treatment group, which contained 4 mice. The mice were euthanized after 3 weeks and β-galactosidase activity was compared to a PBS control, with a p-value<0.05 indicating significance.

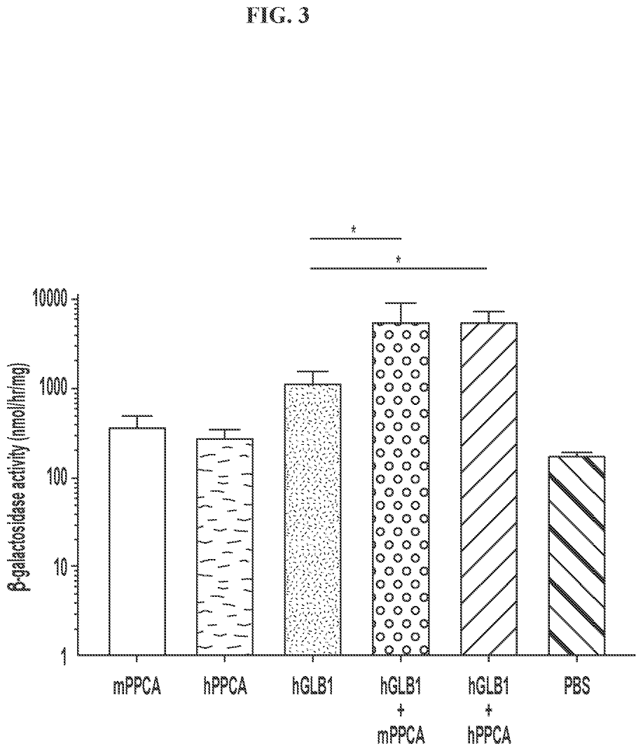

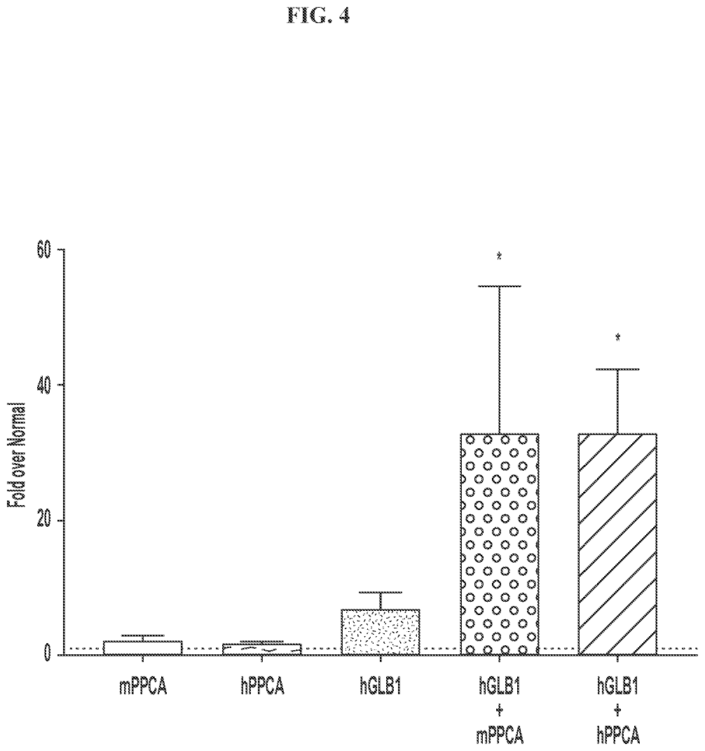

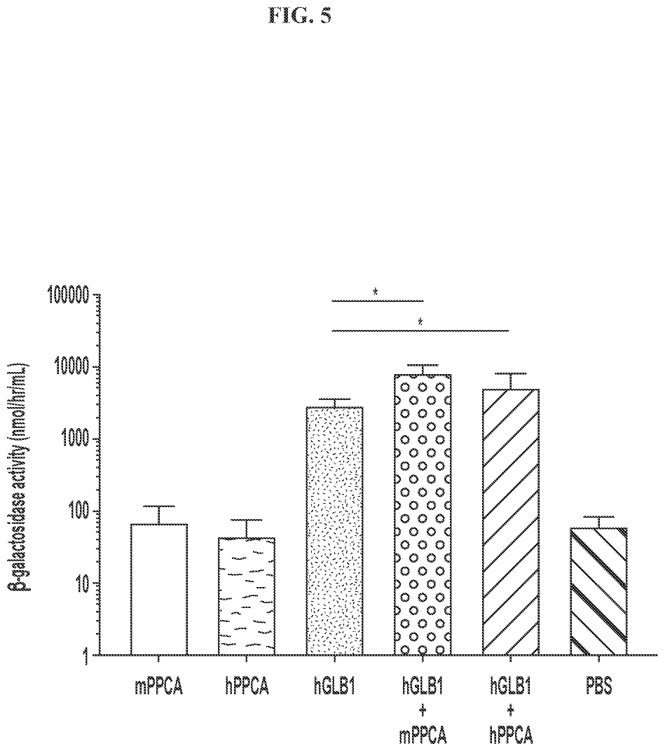

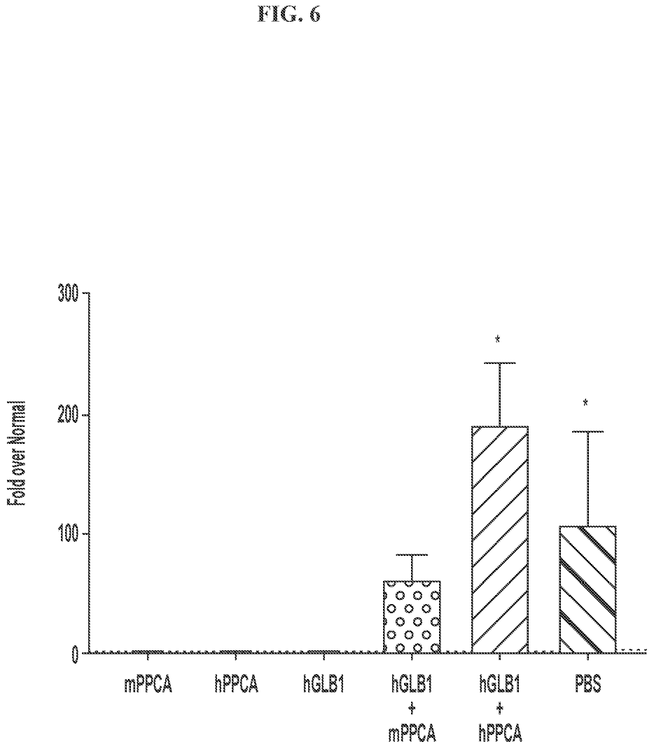

β-galactosidase activity was measured in liver (FIGS. 3-4) and serum (FIGS. 5-6) to investigate whether co-expression of β-galactosidase and Cathepsin A results in increased β-galactosidase enzyme activity. Although expression of human β-galactosidase increased liver enzyme activity (FIG. 3), the co-expression of either mouse or human Cathepsin A with human β-galactosidase significantly increased liver β-galactosidase enzyme activity (FIG. 4). Similarly, expression of human β-galactosidase increased serum enzyme activity (FIG. 5), and co-expression of either mouse or human Cathepsin A with human β-galactosidase increased β-galactosidase enzyme activity over 40-fold compared with PBS (control) (FIG. 6).

### Example 3: Increased Expression of Mature/β-Galactosidase Expression in Liver by Co-Administration of AAV9 Vectors Encoding GLB1 and Cathepsin A

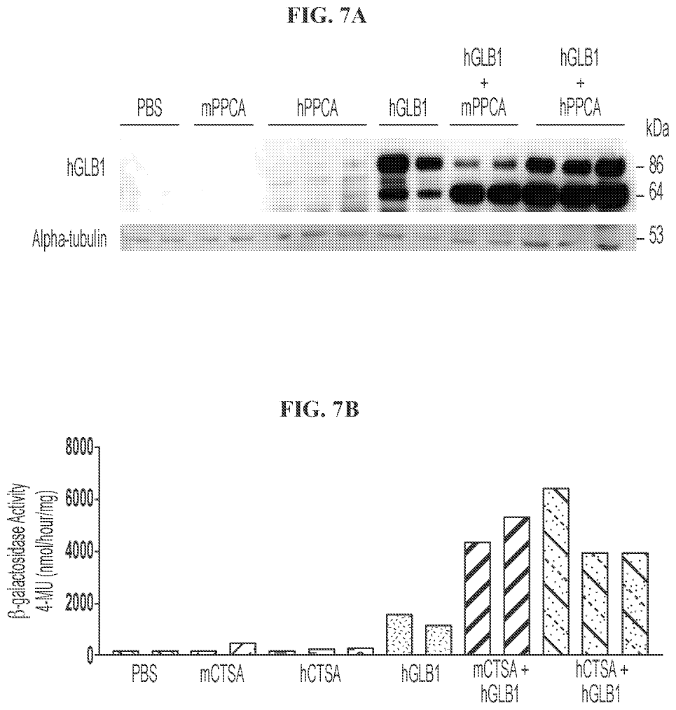

Total and mature hGLB1 was measured from liver of C57BL/6 mice injected with: (1) PBS, (2) mouse PPCA, (3) human PPCA, (4) human GLB1, (5) human GLB1+ mouse PPCA, or (6) human GLB1+ human PPCA. The presence of precursor (86 kilodalton (kDa)) and mature (66 kDa) GLB1 protein was analyzed by western blot with an antibody against human GLB1. The mature protein to precursor protein ratio increased dramatically in animals co-administered human GLB1 and PPCA (either mouse or human) (FIGS. 7A and 7B).

SEQUENCES
nucleic acid sequence of human GLB1
SEQ ID NO: 1
ATGCCGGGGTTCCTGGTTCGCATCCTCCTTCTGCTGCTGGTTCTGCTGCTTCTGGGCC
CTACGCGCGGCTTGCGCAATGCCACCCAGAGGATGTTTGAAATTGACTATAGCCGG
GACTCCTTCCTCAAGGATGGCCAGCCATTTCGCTACATCTCAGGAAGCATTCACTAC
TCCCGTGTGCCCCGCTTCTACTGGAAGGACCGGCTGCTGAAGATGAAGATGGCTGG
GCTGAACGCCATCCAGACGTATGTGCCCTGGAACTTTCATGAGCCCTGGCCAGGAC
AGTACCAGTTTTCTGAGGACCATGATGTGGAATATTTTCTTCGGCTGGCTCATGAGC
TGGGACTGCTGGTTATCCTGAGGCCCGGGCCCTACATCTGTGCAGAGTGGGAAATG
GGAGGATTACCTGCTTGGCTGCTAGAGAAAGAGTCTATTCTTCTCCGCTCCTCCGAC
CCAGATTACCTGGCAGCTGTGGACAAGTGGTTGGGAGTCCTTCTGCCCAAGATGAA
GCCTCTCCTCTATCAGAATGGAGGGCCAGTTATAACAGTGCAGGTTGAAAATGAAT
ATGGCAGCTACTTTGCCTGTGATTTTGACTACCTGCGCTTCCTGCAGAAGCGCTTTC
GCCACCATCTGGGGGATGATGTGGTTCTGTTTACCACTGATGGAGCACATAAAACA
TTCCTGAAATGTGGGGCCCTGCAGGGCCTCTACACCACGGTGGACTTTGGAACAGG
CAGCAACATCACAGATGCTTTCCTAAGCCAGAGGAAGTGTGAGCCCAAAGGACCCT
TGATCAATTCTGAATTCTATACTGGCTGGCTAGATCACTGGGGCCAACCTCACTCCA
CAATCAAGACCGAAGCAGTGGCTTCCTCCCTCTATGATATACTTGCCCGTGGGGCGA
GTGTGAACTTGTACATGTTTATAGGTGGGACCAATTTTGCCTATTGGAATGGGGCCA
ACTCACCCTATGCAGCACAGCCCACCAGCTACGACTATGATGCCCCACTGAGTGAG
GCTGGGGACCTCACTGAGAAGTATTTTGCTCTGCGAAACATCATCCAGAAGTTTGA
AAAAGTACCAGAAGGTCCTATCCCTCCATCTACACCAAAGTTTGCATATGGAAAGG
TCACTTTGGAAAAGTTAAAGACAGTGGGAGCAGCTCTGGACATTCTGTGTCCCTCTG
GGCCCATCAAAAGCCTTTATCCCTTGACATTTATCCAGGTGAAACAGCATTATGGGT
TTGTGCTGTACCGGACAACACTTCCTCAAGATTGCAGCAACCCAGCACCTCTCTCTT
CACCCCTCAATGGAGTCCACGATCGAGCATATGTTGCTGTGGATGGGATCCCCCAG
GGAGTCCTTGAGCGAAACAATGTGATCACTCTGAACATAACAGGGAAAGCTGGAGC
CACTCTGGACCTTCTGGTAGAGAACATGGGACGTGTGAACTATGGTGCATATATCA
ACGATTTTAAGGGTTTGGTTTCTAACCTGACTCTCAGTTCCAATATCCTCACGGACT
GGACGATCTTTCCACTGGACACTGAGGATGCAGTGCGCAGCCACCTGGGGGGCTGG
GGACACCGTGACAGTGGCCACCATGATGAAGCCTGGGCCCACAACTCATCCAACTA
CACGCTCCCGGCCTTTTATATGGGGAACTTCTCCATTCCCAGTGGGATCCCAGACTT
GCCCCAGGACACCTTTATCCAGTTTCCTGGATGGACCAAGGGCCAGGTCTGGATTA
ATGGCTTTAACCTTGGCCGCTATTGGCCAGCCCGGGGCCCTCAGTTGACCTTGTTTG
TGCCCCAGCACATCCTGATGACCTCGGCCCCAAACACCATCACCGTGCTGGAACTG
GAGTGGGCACCCTGCAGCAGTGATGATCCAGAACTATGTGCTGTGACGTTCGTGGA
CAGGCCAGTTATTGGCTCATCTGTGACCTACGATCATCCCTCCAAACCTGTTGAAAA
AAGACTCATGCCCCCACCCCCGCAAAAAAACAAAGATTCATGGCTGGACCATGTAT
GA
nucleic acid sequence of mouse GLB1
SEQ ID NO: 2
ATGCTCCGGGTCCCCCTGTGTACGCCGCTCCCGCTCCTGGCACTGCTGCAACTGCTG
GGCGCTGCGCACGGCATCTATAATGTCACCCAGAGGACATTTAAGCTCGACTACAG
CCGGGACCGCTTCCTCAAGGATGGACAGCCATTCCGATACATCTCGGGAAGCATTC
ATTACTTCCGGATACCCCGCTTCTACTGGGAGGACCGGCTGCTGAAGATGAAGATG
GCTGGGCTGAATGCTATCCAGATGTACGTGCCCTGGAACTTCCATGAACCCCAACC
AGGACAATATGAGTTTTCTGGGGACCGTGATGTGGAGCATTTCATCCAGCTGGCTCA
TGAGCTGGGACTCCTGGTGATCCTGAGGCCTGGGCCCTACATCTGTGCAGAGTGGG
ACATGGGGGGCTTACCTGCTTGGCTACTAGAGAAACAATCTATCGTTCTCCGGTCTT
CTGACCCAGACTACCTTGTAGCTGTGGATAAATGGCTGGCAGTCCTTCTGCCCAAGA
TGAAGCCCCTGCTCTACCAGAACGGAGGACCGATCATAACCGTGCAGGTTGAGAAT
GAGTACGGGTCCTACTTTGCCTGCGATTACGACTACCTACGCTTCCTGGTGCACCGC
TTCCGCTACCATCTGGGTAATGACGTCATTCTCTTCACCACCGACGGAGCAAGTGAA
AAAATGCTGAAGTGTGGGACCCTGCAGGACCTGTACGCCACAGTGGATTTTGGAAC
AGGCAACAATATCACACAAGCTTTCCTGGTCCAGAGGAAGTTTGAACCTAAAGGAC
CTTTGATCAATTCTGAGTTCTATACTGGCTGGCTAGACCACTGGGGTAAACCCCATT
CCACGGTGAAAACTAAAACACTGGCTACCTCCCTCTATAACCTGCTTGCCCGTGGGG
CCAACGTGAACTTGTACATGTTTATAGGTGGGACCAATTTTGCCTATTGGAATGGTG
CCAACACGCCCTATGAGCCACAGCCCACCAGCTATGACTACGACGCCCCACTGAGC
GAGGCTGGGGACCTCACTAAGAAGTATTTTGCTCTTCGAGAAGTCATTCAGATGTTT
AAAGAAGTCCCAGAAGGCCCTATCCCTCCGTCTACACCCAAATTTGCATATGGAAA
AGTTGCTCTGAGAAAGTTCAAGACAGTGGCTGAAGCTCTGGGTATCCTGTGTCCCA
ATGGGCCAGTGAAAAGCCTCTATCCCCTGACATTCACTCAGGTAAAACAGTATTTTG
GGTATGTGCTGTACCGAACAACGCTTCCTCAAGATTGCAGTAACCCGAAACCCATTT
TCTCTTCACCCTTCAATGGTGTCCGTGATCGGGCTTACGTCTCTGTGGACGGGGTCC
CCCAAGGAATCCTTGATCGAAACCTCATGACAGCTCTGAACATACGGGGGAAGGCT
GGAGCCACGCTGGACATCCTGGTGGAGAACATGGGGCGTGTGAACTATGGCAGATT
CATCAATGACTTCAAGGGTTTGATTTCCAACATGACTATCAACTCCACTGTCCTCAC
CAACTGGACGGTCTTCCCACTGAACACTGAGGCCATGGTACGCAACCATCTCTGGG
GCCGGGAGGCCAGTGATGAGGGTCACCTTGACGGACGGTCGACCTCCAATTCTTCG
GACCTCATACTCCCCACCTTTTACGTGGGCAACTTCTCCATCCCCTCGGGCATCCCA
GACCTGCCACAGGACACCTTCATCCAGTTTCCTGGGTGGTCCAAGGGTCAAGTATG
GATCAATGGCTTTAACCTCGGCCGATACTGGCCCACAATGGGCCCACAAAAGACCT
TGTTCGTGCCAAGGAACATCCTGACCACTTCAGCCCCAAACAACATCACAGTGTTG
GAGCTAGAGTTTGCACCCTGCAGCGAGGGGACCCCAGAGCTGTGTACAGTAGAGTT
TGTTGACACTCCGGTCATTTCCTGA
nucleic acid sequence of human CTSA
SEQ ID NO: 3
ATGACTTCCAGTCCCCGGGCGCCTCCTGGAGAGCAAGGACGCGGGGGAGCAGAGAT
GATCCGAGCAGCGCCTCCACCACTGTTCTTGTTGCTGCTCCTGTTGCTGTTGCTAGTG
TCCTGGGCGTCCCGAGGCGAGGCAGCCCCCGACCAGGACGAGATCCAGCGCCTCCC
CGGGCTGGCCAAGCAGCCGTCTTTCCGCCAGTACTCCGGCTACCTCAAAGGCTCCG
GCTCCAAGCACCTCCACTACTGGTTTGTGGAGTCCCAGAAGGATCCCGAGAACAGC
CCTGTGGTGCTTTGGCTCAATGGGGGTCCCGGCTGCAGCTCACTAGATGGGCTCCTC
ACAGAGCATGGCCCCTTCCTGGTCCAGCCAGATGGTGTCACCCTGGAGTACAACCC
CTATTCTTGGAATCTGATTGCCAATGTGTTATACCTGGAGTCCCCAGCTGGGGTGGG
CTTCTCCTACTCCGATGACAAGTTTTATGCAACTAATGACACTGAGGTCGCCCAGAG
CAATTTTGAGGCCCTTCAAGATTTCTTCCGCCTCTTTCCGGAGTACAAGAACAACAA
ACTTTTCCTGACCGGGGAGAGCTATGCTGGCATCTACATCCCCACCCTGGCCGTGCT
GGTCATGCAGGATCCCAGCATGAACCTTCAGGGGCTGGCTGTGGGCAATGGACTCT
CCTCCTATGAGCAGAATGACAACTCCCTGGTCTACTTTGCCTACTACCATGGCCTTC
TGGGGAACAGGCTTTGGTCTTCTCTCCAGACCCACTGCTGCTCTCAAAACAAGTGTA
ACTTCTATGACAACAAAGACCTGGAATGCGTGACCAATCTTCAGGAAGTGGCCCGC
ATCGTGGGCAACTCTGGCCTCAACATCTACAATCTCTATGCCCCGTGTGCTGGAGGG
GTGCCCAGCCATTTTAGGTATGAGAAGGACACTGTTGTGGTCCAGGATTTGGGCAA
CATCTTCACTCGCCTGCCACTCAAGCGGATGTGGCATCAGGCACTGCTGCGCTCAGG
GGATAAAGTGCGCATGGACCCCCCCTGCACCAACACAACAGCTGCTTCCACCTACC
TCAACAACCCGTACGTGCGGAAGGCCCTCAACATCCCGGAGCAGCTGCCACAATGG
GACATGTGCAACTTTCTGGTAAACTTACAGTACCGCCGTCTCTACCGAAGCATGAAC
TCCCAGTATCTGAAGCTGCTTAGCTCACAGAAATACCAGATCCTATTATATAATGGA
GATGTAGACATGGCCTGCAATTTCATGGGGGATGAGTGGTTTGTGGATTCCCTCAAC
CAGAAGATGGAGGTGCAGCGCCGGCCCTGGTTAGTGAAGTACGGGGACAGCGGGG
AGCAGATTGCCGGCTTCGTGAAGGAGTTCTCCCACATCGCCTTTCTCACGATCAAGG
GCGCCGGCCACATGGTTCCCACCGACAAGCCCCTCGCTGCCTTCACCATGTTCTCCC
GCTTCCTGAACAAGCAGCCATACTGA
nucleic acid sequence of mouse CTSA
SEQ ID NO: 4
ATGACTTCCAGTCCAAAGGCGCCTCCTGGAGAGCAAGGACGCAAGGAAGCAGAGA
TGCCCGGAACCGCGCTGTCTCCACTGCTCTTGTTGCTGCTCCTGTCCTGGGCGTCCC
GGAACGAAGCAGCTCCGGACCAGGATGAAATCGATTGTCTCCCCGGCCTGGCCAAG
CAGCCCTCTTTCCGGCAATACTCCGGCTACCTCAGAGCATCGGACTCCAAGCACTTC
CACTACTGGTTTGTGGAGTCGCAGAACGACCCAAAGAACAGCCCCGTGGTGCTTTG
GCTTAACGGGGGTCCCGGCTGCAGCTCGCTCGATGGGCTGCTTACAGAGCACGGCC
CCTTTCTGATCCAGCCAGATGGTGTCACCCTGGAGTACAACCCCTATGCTTGGAACC
TGATTGCCAACGTGCTGTATATCGAGTCCCCAGCTGGGGTGGGCTTCTCCTACTCGG
ATGACAAGATGTACGTGACCAATGACACAGAGGTGGCGGAGAACAATTATGAAGC
CCTTAAAGACTTCTTCCGCCTCTTTCCGGAATACAAGGACAACAAACTTTTCCTGAC
AGGAGAGAGCTATGCTGGCATCTACATCCCCACCTTGGCTGTACTGGTCATGCAGG
ATCCTAGCATGAATCTTCAGGGGCTGGCTGTGGGCAATGGACTTGCCTCCTATGAGC
AGAACGACAACTCCCTGGTCTACTTTGCCTACTACCATGGCCTTCTGGGGAACAGAC
TTTGGACTTCACTGCAGACCCACTGCTGCGCTCAGAACAAGTGTAACTTCTATGACA
ACAAAGACCCAGAGTGTGTAAACAATCTCCTGGAAGTGTCTCGAATTGTGGGCAAA
TCTGGCCTCAACATCTACAATCTCTATGCTCCGTGTGCTGGTGGGGTGCCCGGCAGA
CATAGATATGAGGACACACTTGTAGTCCAGGATTTTGGCAACATCTTCACTCGCCTG
CCACTTAAGCGGAGATTTCCTGAGGCACTGATGCGTTCTGGGGACAAGGTACGCTT
GGATCCTCCCTGCACCAACACCACAGCCCCTTCCAACTACCTCAACAACCCCTATGT
TCGGAAGGCTCTCCACATCCCCGAGTCGCTGCCCCGCTGGGACATGTGCAACTTCTT
GGTGAATTTACAGTACCGCCGCCTCTACCAAAGCATGAACTCCCAGTACCTGAAGC
TGCTCAGTTCACAGAAATACCAGATCCTGCTCTACAACGGAGATGTGGACATGGCC
TGCAACTTCATGGGCGATGAGTGGTTTGTGGATTCGCTCAACCAGAAGATGGAGGT
GCAGCGCCGGCCCTGGCTAGTGGACTACGGGGAGAGCGGAGAACAGGTAGCTGGT
TTCGTGAAGGAGTGTTCACACATCACCTTCCTCACCATCAAGGGTGCCGGACACATG
GTCCCCACGGACAAGCCTCGAGCTGCTTTTACCATGTTCTCGAGGTTCCTGAACAAA
GAGCCTTACTGA
amino acid sequence of human β-galactosidase
SEQ ID NO: 5
MPGFLVRILPLLLVLLLLGPTRGLRNATQRMFEIDYSRDSFLKDGQPFRYISGSIHYSRV
PRFYWKDRLLKMKMAGLNAIQTYVPWNFHEPWPGQYQFSEDHDVEYFLRLAHELGLL
VILRPGPYICAEWEMGGLPAWLLEKESILLRSSDPDYLAAVDKWLGVLLPKMKPLLYQ
NGGPVITVQVENEYGSYFACDFDYLRFLQKRFRHHLGDDVVLFTTDGAHKTFLKCGAL
QGLYTTVDFGTGSNITDAFLSQRKCEPKGPLINSEFYTGWLDHWGQPHSTIKTEAVASS
LYDILARGASVNLYMFIGGTNFAYWNGANSPYAAQPTSYDYDAPLSEAGDLTEKYFAL
RNIIQKFEKVPEGPIPPSTPKFAYGKVTLEKLKTVGAALDILCPSGPIKSLYPLTFIQVKQH
YGFVLYRTTLPQDCSNPAPLSSPLNGVHDRAYVAVDGIPQGVLERNNVITLNITGKAGA
TLDLLVENMGRVNYGAYINDFKGLVSNLTLSSNILTDWTIFPLDTEDAVRSHLGGWGH
RDSGHHDEAWAHNSSNYTLPAFYMGNFSIPSGIPDLPQDTFIQFPGWTKGQVWINGFNL
GRYWPARGPQLTLFVPQHILMTSAPNTITVLELEWAPCSSDDPELCAVTFVDRPVIGSSV
TYDHPSKPVEKRLMPPPPQKNKDSWLDHV
amino acid sequence of mouse β-galactosidase
SEQ ID NO: 6
MLRVPLCTPLPLLALLQLLGAAHGIYNVTQRTFKLDYSRDRFLKDGQPFRYISGSIHYFR
IPRFYWEDRLLKMKMAGLNAIQMYVPWNFHEPQPGQYEFSGDRDVEHFIQLAHELGLL
VILRPGPYICAEWDMGGLPAWLLEKQSIVLRSSDPDYLVAVDKWLAVLLPKMKPLLYQ
NGGPIITVQVENEYGSYFACDYDYLRFLVHRFRYHLGNDVILFTTDGASEKMLKCGTLQ
DLYATVDFGTGNNITQAFLVQRKFEPKGPLINSEFYTGWLDHWGKPHSTVKTKTLATSL
YNLLARGANVNLYMFIGGTNFAYWNGANTPYEPQPTSYDYDAPLSEAGDLTKKYFAL
REVIQMFKEVPEGPIPPSTPKFAYGKVALRKFKTVAEALGILCPNGPVKSLYPLTFTQVK
QYFGYVLYRTTLPQDCSNPKPIFSSPFNGVRDRAYVSVDGVPQGILDRNLMTALNIRGK
AGATLDILVENMGRVNYGRFINDFKGLISNMTINSTVLTNWTVFPLNTEAMVRNHLWG
REASDEGHLDGRSTSNSSDLILPTFYVGNFSIPSGIPDLPQDTFIQFPGWSKGQVWINGFN
LGRYWPTMGPQKTLFVPRNILTTSAPNNITVLELEFAPCSEGTPELCTVEFVDTPVIS
amino acid sequence of human Cathepsin A
SEQ ID NO: 7
MTSSPRAPPGEQGRGGAEMIRAAPPPLFLLLLLLLLLVSWASRGEAAPDQDEIQRLPGLA
KQPSFRQYSGYLKGSGSKHLHYWFVESQKDPENSPVVLWLNGGPGCSSLDGLLTEHGP
FLVQPDGVTLEYNPYSWNLIANVLYLESPAGVGFSYSDDKFYATNDTEVAQSNFEALQ
DFFRLFPEYKNNKLFLTGESYAGIYIPTLAVLVMQDPSMNLQGLAVGNGLSSYEQNDNS
LVYFAYYHGLLGNRLWSSLQTHCCSQNKCNFYDNKDLECVTNLQEVARIVGNSGLNIY
NLYAPCAGGVPSHFRYEKDTVVVQDLGNIFTRLPLKRMWHQALLRSGDKVRMDPPCT
NTTAASTYLNNPYVRKALNIPEQLPQWDMCNFLVNLQYRRLYRSMNSQYLKLLSSQK
YQILLYNGDVDMACNFMGDEWFVDSLNQKMEVQRRPWLVKYGDSGEQIAGFVKEFS
HIAFLTIKGAGHMVPTDKPLAAFTMFSRFLNKQPY
amino acid sequence of mouse Cathepsin A
SEQ ID NO: 8
MTSSPKAPPGEQGRKEAEMPGTALSPLLLLLLLSWASRNEAAPDQDEIDCLPGLAKQPS
FRQYSGYLRASDSKHFHYWFVESQNDPKNSPVVLWLNGGPGCSSLDGLLTEHGPFLIQP
DGVTLEYNPYAWNLIANVLYIESPAGVGFSYSDDKMYVTNDTEVAENNYEALKDFFRL
FPEYKDNKLFLTGESYAGIYIPTLAVLVMQDPSMNLQGLAVGNGLASYEQNDNSLVYF
AYYHGLLGNRLWTSLQTHCCAQNKCNFYDNKDPECVNNLLEVSRIVGKSGLNIYNLY
APCAGGVPGRHRYEDTLVVQDFGNIFTRLPLKRRFPEALMRSGDKVRLDPPCTNTTAPS
NYLNNPYVRKALHIPESLPRWDMCNFLVNLQYRRLYQSMNSQYLKLLSSQKYQILLYN
GDVDMACNFMGDEWFVDSLNQKMEVQRRPWLVDYGESGEQVAGFVKECSHITFLTIK
GAGHMVPTDKPRAAFTMFSRFLNKEPY
nucleic acid sequence of pAAV-CB-human GLB1-expression vector
SEQ ID NO: 9
CCTGCAGGCAGCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGG
AGTGGCCAACTCCATCACTAGGGGTTCCTGCGGCCAGATCTTCAATATTGGCCATTA
GCCATATTATTCATTGGTTATATAGCATAAATCAATATTGGCTATTGGCCATTGCAT
ACGTTGTATCTATATCATAATATGTACATTTATATTGGCTCATGTCCAATATGACCG
CCATGTTGGCATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAAC
TGCCCACTTGGCAGTACATCAAGTGTATCATATGCCAAGTCCGCCCCCTATTGACGT
CAATGACGGTAAATGGCCCGCCTGGCATTATGCCCAGTACATGACCTTACGGGACT
TTCCTACTTGGCAGTACATCTACGTATTAGTCATCGCTATTACCATGGTCGAGGTGA
GCCCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGC
GCGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGG
GCTGTAATTAGCGCTTGGTTTAATGACGGCTTGTTTCTTTTCTGTGGCTGCGTGAAA
GCCTTGAGGGGCTCCGGGAGGGCCCTTTGTGCGGGGGGAGCGGCTCGGGGGGTGCG
TGCGTGTGTGTGTGCGTGGGGAGCGCCGCGTGCGGCTCCGCGCTGCCCGGCGGCTG
TGAGCGCTGCGGGCGCGGCGCGGGGCTTTGTGCGCTCCGCAGTGTGCGCGAGGGGA
GCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGTCGGTCG
GGCTGCAACCCCCCCTGCACCCCCCTCCCCGAGTTGCTGAGCACGGCCCGGCTTCGG
GTGCGGGGCTCCGTACGGGGCGTGGCGCGGGGCTCGCCGTGCCGGGCGGGGGGTG
GCGGCAGGTGGGGGTGCCGGGCGGGGCGGGGCCGCCTCGGGCCGGGGAGGGCTCG
GGGGAGGGGCGCGGCGGCCCCCGGAGCGCCGGCGGCTGTCGAGGCGCGGCGAGCC
GCAGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGCGC
GGGGCGAAGCGGTGCGGCGCCGGCAGGAAGGAAATGGGCGGGGAGGGCCTTCGTG
CGTCGCCGCGCCGCCGTCCCCTTCTCCCTCTCCAGCCTCGGGGCTGTCCGCGGGGGG
ACGGCTGCCTTCGGGGGGGACGGGGCAGGGCGGGGTTCGGCTTCTGGCGTGTGACC
GGCGGCTCTAGAGCCTCTGCTAACCATGTTCATGCCTTCTTCTTTTTCCTACAGCTCC
TGGGCAACGTGCTGGTTATTGTGCTGTCTCATCATTTTGGCAAAGAATTCGATATCA
AGCTTGCTAGCGCCACCATGCCGGGGTTCCTGGTTCGCATCCTCCTTCTGCTGCTGG
TTCTGCTGCTTCTGGGCCCTACGCGCGGCTTGCGCAATGCCACCCAGAGGATGTTTG
AAATTGACTATAGCCGGGACTCCTTCCTCAAGGATGGCCAGCCATTTCGCTACATCT
CAGGAAGCATTCACTACTCCCGTGTGCCCCGCTTCTACTGGAAGGACCGGCTGCTGA
AGATGAAGATGGCTGGGCTGAACGCCATCCAGACGTATGTGCCCTGGAACTTTCAT
GAGCCCTGGCCAGGACAGTACCAGTTTTCTGAGGACCATGATGTGGAATATTTTCTT
CGGCTGGCTCATGAGCTGGGACTGCTGGTTATCCTGAGGCCCGGGCCCTACATCTGT
GCAGAGTGGGAAATGGGAGGATTACCTGCTTGGCTGCTAGAGAAAGAGTCTATTCT
TCTCCGCTCCTCCGACCCAGATTACCTGGCAGCTGTGGACAAGTGGTTGGGAGTCCT
TCTGCCCAAGATGAAGCCTCTCCTCTATCAGAATGGAGGGCCAGTTATAACAGTGC
AGGTTGAAAATGAATATGGCAGCTACTTTGCCTGTGATTTTGACTACCTGCGCTTCC
TGCAGAAGCGCTTTCGCCACCATCTGGGGGATGATGTGGTTCTGTTTACCACTGATG
GAGCACATAAAACATTCCTGAAATGTGGGGCCCTGCAGGGCCTCTACACCACGGTG
GACTTTGGAACAGGCAGCAACATCACAGATGCTTTCCTAAGCCAGAGGAAGTGTGA
GCCCAAAGGACCCTTGATCAATTCTGAATTCTATACTGGCTGGCTAGATCACTGGGG
CCAACCTCACTCCACAATCAAGACCGAAGCAGTGGCTTCCTCCCTCTATGATATACT
TGCCCGTGGGGCGAGTGTGAACTTGTACATGTTTATAGGTGGGACCAATTTTGCCTA
TTGGAATGGGGCCAACTCACCCTATGCAGCACAGCCCACCAGCTACGACTATGATG
CCCCACTGAGTGAGGCTGGGGACCTCACTGAGAAGTATTTTGCTCTGCGAAACATC
ATCCAGAAGTTTGAAAAAGTACCAGAAGGTCCTATCCCTCCATCTACACCAAAGTTT
GCATATGGAAAGGTCACTTTGGAAAAGTTAAAGACAGTGGGAGCAGCTCTGGACAT
TCTGTGTCCCTCTGGGCCCATCAAAAGCCTTTATCCCTTGACATTTATCCAGGTGAA
ACAGCATTATGGGTTTGTGCTGTACCGGACAACACTTCCTCAAGATTGCAGCAACCC
AGCACCTCTCTCTTCACCCCTCAATGGAGTCCACGATCGAGCATATGTTGCTGTGGA
TGGGATCCCCCAGGGAGTCCTTGAGCGAAACAATGTGATCACTCTGAACATAACAG
GGAAAGCTGGAGCCACTCTGGACCTTCTGGTAGAGAACATGGGACGTGTGAACTAT
GGTGCATATATCAACGATTTTAAGGGTTTGGTTTCTAACCTGACTCTCAGTTCCAAT
ATCCTCACGGACTGGACGATCTTTCCACTGGACACTGAGGATGCAGTGCGCAGCCA
CCTGGGGGGCTGGGGACACCGTGACAGTGGCCACCATGATGAAGCCTGGGCCCACA
ACTCATCCAACTACACGCTCCCGGCCTTTTATATGGGGAACTTCTCCATTCCCAGTG
GGATCCCAGACTTGCCCCAGGACACCTTTATCCAGTTTCCTGGATGGACCAAGGGCC
AGGTCTGGATTAATGGCTTTAACCTTGGCCGCTATTGGCCAGCCCGGGGCCCTCAGT
TGACCTTGTTTGTGCCCCAGCACATCCTGATGACCTCGGCCCCAAACACCATCACCG
TGCTGGAACTGGAGTGGGCACCCTGCAGCAGTGATGATCCAGAACTATGTGCTGTG
ACGTTCGTGGACAGGCCAGTTATTGGCTCATCTGTGACCTACGATCATCCCTCCAAA
CCTGTTGAAAAAAGACTCATGCCCCCACCCCCGCAAAAAAACAAAGATTCATGGCT
GGACCATGTATGACTCGAGTTTTTTTTTGCGGCCGCTTCGAGCAGACATGATAAGAT
ACATTGATGAGTTTGGACAAACCACAACTAGAATGCAGTGAAAAAAATGCTTTATT
TGTGAAATTTGTGATGCTATTGCTTTATTTGTAACCATTATAAGCTGCAATAAACAA
GTTAACAACAACAATTGCATTCATTTTATGTTTCAGGTTCAGGGGGAGATGTGGGAG
GTTTTTTAAAGCAAGTAAAACCTCTACAAATGTGGTAAAATCGATAGGCCGCAGGA
ACCCCTAGTGATGGAGTTGGCCACTCCCTCTCTGCGCGCTCGCTCGCTCACTGAGGC
CGGGCGACCAAAGGTCGCCCGACGCCCGGGCGGCCTCAGTGAGCGAGCGAGCGCG
CAGCTGCCTGCAGGACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACA
CTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAAT
CAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTG
CAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCA
GTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGC
TTCATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACCCAAC
TGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCG
AAAAGTGCCACCTGACGTCTAAGAAACCATTATTATCATGACATTAACCTATAAAA
ATAGGCGTATCACGAGGCCCTTTCGTCTCGCGCGTTTCGGTGATGACGGTGAAAACC
TCTGACACATGCAGCTCCCGGAGACGGTCACAGCTTGTCTGTAAGCGGATGCCGGG
AGCAGACAAGCCCGTCAGGGCGCGTCAGCGGGTGTTGGCGGGTGTCGGGGCTGGCT
TAACTATGCGGCATCAGAGCAGATTGTACTGAGAGTGCACCATAAAATTGTAAACG
TTAATATTTTGTTAAAATTCGCGTTAAATTTTTGTTAAATCAGCTCATTTTTTAACCA
ATAGACCGAAATCGGCAAAATCCCTTATAAATCAAAAGAATAGCCCGAGATAGAGT
TGAGTGTTGTTCCAGTTTGGAACAAGAGTCCACTATTAAAGAACGTGGACTCCAAC
GTCAAAGGGCGAAAAACCGTCTATCAGGGCGATGGCCCACTACGTGAACCATCACC
CAAATCAAGTTTTTTGGGGTCGAGGTGCCGTAAAGCACTAAATCGGAACCCTAAAG
GGAGCCCCCGATTTAGAGCTTGACGGGGAAAGCCGGCGAACGTGGCGAGAAAGGA
AGGGAAGAAAGCGAAAGGAGCGGGCGCTAAGGCGCTGGCAAGTGTAGCGGTCACG
CTGCGCGTAACCACCACACCCGCCGCGCTTAATGCGCCGCTACAGGGCGCGTACTA
TGGTTGCTTTGACGTATGCGGTGTGAAATACCGCACAGATGCGTAAGGAGAAAATA
CCGCATCAGGCGCC
nucleic acid sequence of pAAV-CB-mouse GLB1-expression vector
SEQ ID NO: 10
CCTGCAGGCAGCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGG
AGTGGCCAACTCCATCACTAGGGGTTCCTGCGGCCAGATCTTCAATATTGGCCATTA
GCCATATTATTCATTGGTTATATAGCATAAATCAATATTGGCTATTGGCCATTGCAT
ACGTTGTATCTATATCATAATATGTACATTTATATTGGCTCATGTCCAATATGACCG
CCATGTTGGCATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAAC
TGCCCACTTGGCAGTACATCAAGTGTATCATATGCCAAGTCCGCCCCCTATTGACGT
CAATGACGGTAAATGGCCCGCCTGGCATTATGCCCAGTACATGACCTTACGGGACT
TTCCTACTTGGCAGTACATCTACGTATTAGTCATCGCTATTACCATGGTCGAGGTGA
GCCCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGC
GCGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGG
GCTGTAATTAGCGCTTGGTTTAATGACGGCTTGTTTCTTTTCTGTGGCTGCGTGAAA
GCCTTGAGGGGCTCCGGGAGGGCCCTTTGTGCGGGGGGAGCGGCTCGGGGGGTGCG
TGCGTGTGTGTGTGCGTGGGGAGCGCCGCGTGCGGCTCCGCGCTGCCCGGCGGCTG
TGAGCGCTGCGGGCGCGGCGCGGGGCTTTGTGCGCTCCGCAGTGTGCGCGAGGGGA
GCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGTCGGTCG
GGCTGCAACCCCCCCTGCACCCCCCTCCCCGAGTTGCTGAGCACGGCCCGGCTTCGG
GTGCGGGGCTCCGTACGGGGCGTGGCGCGGGGCTCGCCGTGCCGGGCGGGGGGTG
GCGGCAGGTGGGGGTGCCGGGCGGGGCGGGGCCGCCTCGGGCCGGGGAGGGCTCG
GGGGAGGGGCGCGGCGGCCCCCGGAGCGCCGGCGGCTGTCGAGGCGCGGCGAGCC
GCAGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGCGC
GGGGCGAAGCGGTGCGGCGCCGGCAGGAAGGAAATGGGCGGGGAGGGCCTTCGTG
CGTCGCCGCGCCGCCGTCCCCTTCTCCCTCTCCAGCCTCGGGGCTGTCCGCGGGGGG
ACGGCTGCCTTCGGGGGGGACGGGGCAGGGCGGGGTTCGGCTTCTGGCGTGTGACC
GGCGGCTCTAGAGCCTCTGCTAACCATGTTCATGCCTTCTTCTTTTTCCTACAGCTCC
TGGGCAACGTGCTGGTTATTGTGCTGTCTCATCATTTTGGCAAAGAATTCGATATCA
AGCTTGCTAGCCCACCATGCTCCGGGTCCCCCTGTGTACGCCGCTCCCGCTCCTGGC
ACTGCTGCAACTGCTGGGCGCTGCGCACGGCATCTATAATGTCACCCAGAGGACAT
TTAAGCTCGACTACAGCCGGGACCGCTTCCTCAAGGATGGACAGCCATTCCGATAC
ATCTCGGGAAGCATTCATTACTTCCGGATACCCCGCTTCTACTGGGAGGACCGGCTG
CTGAAGATGAAGATGGCTGGGCTGAATGCTATCCAGATGTACGTGCCCTGGAACTT
CCATGAACCCCAACCAGGACAATATGAGTTTTCTGGGGACCGTGATGTGGAGCATT
TCATCCAGCTGGCTCATGAGCTGGGACTCCTGGTGATCCTGAGGCCTGGGCCCTACA
TCTGTGCAGAGTGGGACATGGGGGGCTTACCTGCTTGGCTACTAGAGAAACAATCT
ATCGTTCTCCGGTCTTCTGACCCAGACTACCTTGTAGCTGTGGATAAATGGCTGGCA
GTCCTTCTGCCCAAGATGAAGCCCCTGCTCTACCAGAACGGAGGACCGATCATAAC
CGTGCAGGTTGAGAATGAGTACGGGTCCTACTTTGCCTGCGATTACGACTACCTACG
CTTCCTGGTGCACCGCTTCCGCTACCATCTGGGTAATGACGTCATTCTCTTCACCACC
GACGGAGCAAGTGAAAAAATGCTGAAGTGTGGGACCCTGCAGGACCTGTACGCCA
CAGTGGATTTTGGAACAGGCAACAATATCACACAAGCTTTCCTGGTCCAGAGGAAG
TTTGAACCTAAAGGACCTTTGATCAATTCTGAGTTCTATACTGGCTGGCTAGACCAC
TGGGGTAAACCCCATTCCACGGTGAAAACTAAAACACTGGCTACCTCCCTCTATAA
CCTGCTTGCCCGTGGGGCCAACGTGAACTTGTACATGTTTATAGGTGGGACCAATTT
TGCCTATTGGAATGGTGCCAACACGCCCTATGAGCCACAGCCCACCAGCTATGACT
ACGACGCCCCACTGAGCGAGGCTGGGGACCTCACTAAGAAGTATTTTGCTCTTCGA
GAAGTCATTCAGATGTTTAAAGAAGTCCCAGAAGGCCCTATCCCTCCGTCTACACCC
AAATTTGCATATGGAAAAGTTGCTCTGAGAAAGTTCAAGACAGTGGCTGAAGCTCT
GGGTATCCTGTGTCCCAATGGGCCAGTGAAAAGCCTCTATCCCCTGACATTCACTCA
GGTAAAACAGTATTTTGGGTATGTGCTGTACCGAACAACGCTTCCTCAAGATTGCAG
TAACCCGAAACCCATTTTCTCTTCACCCTTCAATGGTGTCCGTGATCGGGCTTACGT
CTCTGTGGACGGGGTCCCCCAAGGAATCCTTGATCGAAACCTCATGACAGCTCTGA
ACATACGGGGGAAGGCTGGAGCCACGCTGGACATCCTGGTGGAGAACATGGGGCG
TGTGAACTATGGCAGATTCATCAATGACTTCAAGGGTTTGATTTCCAACATGACTAT
CAACTCCACTGTCCTCACCAACTGGACGGTCTTCCCACTGAACACTGAGGCCATGGT
ACGCAACCATCTCTGGGGCCGGGAGGCCAGTGATGAGGGTCACCTTGACGGACGGT
CGACCTCCAATTCTTCGGACCTCATACTCCCCACCTTTTACGTGGGCAACTTCTCCAT
CCCCTCGGGCATCCCAGACCTGCCACAGGACACCTTCATCCAGTTTCCTGGGTGGTC
CAAGGGTCAAGTATGGATCAATGGCTTTAACCTCGGCCGATACTGGCCCACAATGG
GCCCACAAAAGACCTTGTTCGTGCCAAGGAACATCCTGACCACTTCAGCCCCAAAC
AACATCACAGTGTTGGAGCTAGAGTTTGCACCCTGCAGCGAGGGGACCCCAGAGCT
GTGTACAGTAGAGTTTGTTGACACTCCGGTCATTTCCTGACTCGAGTTTTTTTTTGCG
GCCGCTTCGAGCAGACATGATAAGATACATTGATGAGTTTGGACAAACCACAACTA
GAATGCAGTGAAAAAAATGCTTTATTTGTGAAATTTGTGATGCTATTGCTTTATTTG
TAACCATTATAAGCTGCAATAAACAAGTTAACAACAACAATTGCATTCATTTTATGT
TTCAGGTTCAGGGGGAGATGTGGGAGGTTTTTTAAAGCAAGTAAAACCTCTACAAA
TGTGGTAAAATCGATAGGCCGCAGGAACCCCTAGTGATGGAGTTGGCCACTCCCTC
TCTGCGCGCTCGCTCGCTCACTGAGGCCGGGCGACCAAAGGTCGCCCGACGCCCGG
GCGGCCTCAGTGAGCGAGCGAGCGCGCAGCTGCCTGCAGGACATGTGAGCAAAAG
GCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGC
TCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGA
ACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACC
ATTATTATCATGACATTAACCTATAAAAATAGGCGTATCACGAGGCCCTTTCGTCTC
GCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAG
CGGGTGTTGGCGGGTGTCGGGGCTGGCTTAACTATGCGGCATCAGAGCAGATTGTA
CTGAGAGTGCACCATAAAATTGTAAACGTTAATATTTTGTTAAAATTCGCGTTAAAT
TTTTGTTAAATCAGCTCATTTTTTAACCAATAGACCGAAATCGGCAAAATCCCTTAT
AAATCAAAAGAATAGCCCGAGATAGAGTTGAGTGTTGTTCCAGTTTGGAACAAGAG
TCCACTATTAAAGAACGTGGACTCCAACGTCAAAGGGCGAAAAACCGTCTATCAGG
GCGATGGCCCACTACGTGAACCATCACCCAAATCAAGTTTTTTGGGGTCGAGGTGC
CGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGC
TAAGGCGCTGGCAAGTGTAGCGGTCACGCTGCGCGTAACCACCACACCCGCCGCGC
TTAATGCGCCGCTACAGGGCGCGTACTATGGTTGCTTTGACGTATGCGGTGTGAAAT
ACCGCACAGATGCGTAAGGAGAAAATACCGCATCAGGCGCC
nucleic acid sequence of pAAV-CB-human CTSA expression vector
SEQ ID NO: 11
GGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAG
AGAGGGAGTGGCCAACTCCATCACTAGGGGTTCCTAGATCTTCAATATTGGCCATTA
GCCATATTATTCATTGGTTATATAGCATAAATCAATATTGGCTATTGGCCATTGCAT
ACGTTGTATCTATATCATAATATGTACATTTATATTGGCTCATGTCCAATATGACCG
CCATGTTGGCATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAAC
TGCCCACTTGGCAGTACATCAAGTGTATCATATGCCAAGTCCGCCCCCTATTGACGT
CAATGACGGTAAATGGCCCGCCTGGCATTATGCCCAGTACATGACCTTACGGGACT
TTCCTACTTGGCAGTACATCTACGTATTAGTCATCGCTATTACCATGGTCGAGGTGA
GCCCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGC
GACGCTGCCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCTCCTCCGG
GCTGTAATTAGCGCTTGGTTTAATGACGGCTTGTTTCTTTTCTGTGGCTGCGTGAAA
GCCTTGAGGGGCTCCGGGAGGGCCCTTTGTGCGGGGGGGAGCGGCTCGGGGGGTGC
GTGCGTGTGTGTGTGCGTGGGGAGCGCCGCGTGCGGCCCGCGCTGCCCGGCGGCTG
TGAGCGCTGCGGGCGCGGCGCGGGGCTTTGTGCGCTCCGCAGTGTGCGCGAGGGGA
GCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGGCGGTC
GGGCTGTAACCCCCCCCTGCACCCCCCTCCCCGAGTTGCTGAGCACGGCCCGGCTTC
GGGTGCGGGGCTCCGTACGGGGCGTGGCGCGGGGCTCGCCGTGCCGGGCGGGGGG
TGGCGGCAGGTGGGGGTGCCGGGCGGGGCGGGGCCGCCTCGGGCCGGGGAGGGCT
CGGGGGAGGGGCGCGGCGGCCCCCGGAGCGCCGGCGGCTGTCGAGGCGCGGCGAG
CCGCAGCCATTGCCTTTTATGGTAATCGTGCGAGAGGGCGCAGGGACTTCCTTTGTC
CCAAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGCCTTCG
TGCGTCGCCGCGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCTGGCGTGTGA
CCGGCGGCTCTAGAGCCTCTGCTAACCATGTTCATGCCTTCTTCTTTTTCCTACAGCT
CCTGGGCAACGTGCTGGTTATTGTGCTGTCTCATCATTTTGGCAAAGAATTCGATAT
CAAGCTTGCTAGCGCCACCATGACTTCCAGTCCCCGGGCGCCTCCTGGAGAGCAAG
GACGCGGGGGAGCAGAGATGATCCGAGCAGCGCCTCCACCACTGTTCTTGTTGCTG
CTCCTGTTGCTGTTGCTAGTGTCCTGGGCGTCCCGAGGCGAGGCAGCCCCCGACCAG
GACGAGATCCAGCGCCTCCCCGGGCTGGCCAAGCAGCCGTCTTTCCGCCAGTACTC
CGGCTACCTCAAAGGCTCCGGCTCCAAGCACCTCCACTACTGGTTTGTGGAGTCCCA
GAAGGATCCCGAGAACAGCCCTGTGGTGCTTTGGCTCAATGGGGGTCCCGGCTGCA
GCTCACTAGATGGGCTCCTCACAGAGCATGGCCCCTTCCTGGTCCAGCCAGATGGTG
TCACCCTGGAGTACAACCCCTATTCTTGGAATCTGATTGCCAATGTGTTATACCTGG
AGTCCCCAGCTGGGGTGGGCTTCTCCTACTCCGATGACAAGTTTTATGCAACTAATG
ACACTGAGGTCGCCCAGAGCAATTTTGAGGCCCTTCAAGATTTCTTCCGCCTCTTTC
CGGAGTACAAGAACAACAAACTTTTCCTGACCGGGGAGAGCTATGCTGGCATCTAC
ATCCCCACCCTGGCCGTGCTGGTCATGCAGGATCCCAGCATGAACCTTCAGGGGCT
GGCTGTGGGCAATGGACTCTCCTCCTATGAGCAGAATGACAACTCCCTGGTCTACTT
TGCCTACTACCATGGCCTTCTGGGGAACAGGCTTTGGTCTTCTCTCCAGACCCACTG
CTGCTCTCAAAACAAGTGTAACTTCTATGACAACAAAGACCTGGAATGCGTGACCA
ATCTTCAGGAAGTGGCCCGCATCGTGGGCAACTCTGGCCTCAACATCTACAATCTCT
ATGCCCCGTGTGCTGGAGGGGTGCCCAGCCATTTTAGGTATGAGAAGGACACTGTT
GTGGTCCAGGATTTGGGCAACATCTTCACTCGCCTGCCACTCAAGCGGATGTGGCAT
CAGGCACTGCTGCGCTCAGGGGATAAAGTGCGCATGGACCCCCCCTGCACCAACAC
AACAGCTGCTTCCACCTACCTCAACAACCCGTACGTGCGGAAGGCCCTCAACATCC
CGGAGCAGCTGCCACAATGGGACATGTGCAACTTTCTGGTAAACTTACAGTACCGC
CGTCTCTACCGAAGCATGAACTCCCAGTATCTGAAGCTGCTTAGCTCACAGAAATAC
CAGATCCTATTATATAATGGAGATGTAGACATGGCCTGCAATTTCATGGGGGATGA
GTGGTTTGTGGATTCCCTCAACCAGAAGATGGAGGTGCAGCGCCGGCCCTGGTTAG
TGAAGTACGGGGACAGCGGGGAGCAGATTGCCGGCTTCGTGAAGGAGTTCTCCCAC
ATCGCCTTTCTCACGATCAAGGGCGCCGGCCACATGGTTCCCACCGACAAGCCCCTC
GCTGCCTTCACCATGTTCTCCCGCTTCCTGAACAAGCAGCCATACTGACTCGAGCGG
CCGCTTCGAGCAGACATGATAAGATACATTGATGAGTTTGGACAAACCACAACTAG
AATGCAGTGAAAAAAATGCTTTATTTGTGAAATTTGTGATGCTATTGCTTTATTTGT
AACCATTATAAGCTGCAATAAACAAGTTAACAACAACAATTGCATTCATTTTATGTT
TCAGGTTCAGGGGGAGATGTGGGAGGTTTTTTAAAGCAAGTAAAACCTCTACAAAT
GTGGTAAAATCGATAGGCCGCAGGAACCCCTAGTGATGGAGTTGGCCACTCCCTCT
CTGCGCGCTCGCTCGCTCACTGAGGCCGCCCGGGCAAAGCCCGGGCGTCGGGCGAC
CTTTGGTCGCCCGGCCTCAGTGAGCGAGCGAGCGCGCAGAGAGGGAGTGGCCAACC
CCCCCCCCCCCCCCCCTGCAGCCTGGCGTAATAGCGAAGAGGCCCGCACCGATCGC
CCTTCCCAACAGTTGCGTAGCCTGAATGGCGAATGGCGCGACGCGCCCTGTAGCGG
CGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGG
CTTTCCCCGTCAAGCTCTAAATCGGGGGCTCCCTTTAGGGTTCCGATTTAGTGCTTTA
CGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGG
ACTCTTGTTCCAAACTGGAACAACACTCAACCCTATCTCGGTCTATTCTTTTGATTTA
TAAGGGATTTTGCCGATTTCGGCCTATTGGTTAAAAAATGAGCTGATTTAACAAAAA
TTTAACGCGAATTTTAACAAAATATTAACGTTTACAATTTCCTGATGCGGTATTTTCT
CCTTACGCATCTGTGCGGTATTTCACACCGCATATGGTGCACTCTCAGTACAATCTG
CTCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGACGAAAG
GGCCTCGTGATACGCCTATTTTTATAGGTTAATGTCATGATAATAATGGTTTCTTAG
ACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAA
TAATATTGAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCACT
TTTAAAGTTCTGCTATGTGGCGCGGTATTATCCCGTATTGACGCCGGGCAAGAGCAA
CTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAAC
CATGAGTGATAACACTGCGGCCAACTTACTTCTGACAACGATCGGAGGACCGAAGG
AGCTAACCGCTTTTTTGCACAACATGGGGGATCATGTAACTCGCCTTGATCGTTGGG
AACCGGAGCTGAATGAAGCCATACCAAACGACGAGCGTGACACCACGATGCCTGTA
GCAATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGG
GTCTCGCGGTATCATTGCAGCACTGGGGCCAGATGGTAAGCCCTCCCGTATCGTAGT
TATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATA
TACTTTAGATTGATTTAAAACTTCATTTTTAATTTAAAAGGATCTAGGTGAAGATCC
TTTTTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCA
AATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACA
CCGAACTGAGATACCTACAGCGTGAGCATTGAGAAAGCGCCACGCTTCCCGAAGGG
AGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCAC
ATGTTCTTTCCTGCGTTATCCCCTGATTCTGTGGATAACCGTATTACCGCCTTTGAGT
GAGCTGATACCGCTCGCCGCAGCCGAACGACCGAGCGCAGCGAGTCAGTGAGCGA
GGAAGCGGAAGAGCGCCCAATACGCAAACCGCCTCTCCCCGCGCGTTGGCCGATTC
ATTAATGCAGGGCTGCAGGGGGGGGGGGGGGGG
nucleic acid sequence of pAAV-CB-mouse CTSA expression vector
SEQ ID NO: 12
GGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAG
AGAGGGAGTGGCCAACTCCATCACTAGGGGTTCCTAGATCTTCAATATTGGCCATTA
GCCATATTATTCATTGGTTATATAGCATAAATCAATATTGGCTATTGGCCATTGCAT
ACGTTGTATCTATATCATAATATGTACATTTATATTGGCTCATGTCCAATATGACCG
CCATGTTGGCATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAAC
TGCCCACTTGGCAGTACATCAAGTGTATCATATGCCAAGTCCGCCCCCTATTGACGT
CAATGACGGTAAATGGCCCGCCTGGCATTATGCCCAGTACATGACCTTACGGGACT
TTCCTACTTGGCAGTACATCTACGTATTAGTCATCGCTATTACCATGGTCGAGGTGA
GCCCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGC
GACGCTGCCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCTCCTCCGG
GCTGTAATTAGCGCTTGGTTTAATGACGGCTTGTTTCTTTTCTGTGGCTGCGTGAAA
GCCTTGAGGGGCTCCGGGAGGGCCCTTTGTGCGGGGGGGAGCGGCTCGGGGGGTGC
GTGCGTGTGTGTGTGCGTGGGGAGCGCCGCGTGCGGCCCGCGCTGCCCGGCGGCTG
TGAGCGCTGCGGGCGCGGCGCGGGGCTTTGTGCGCTCCGCAGTGTGCGCGAGGGGA
GCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGGCGGTC
GGGCTGTAACCCCCCCCTGCACCCCCCTCCCCGAGTTGCTGAGCACGGCCCGGCTTC
GGGTGCGGGGCTCCGTACGGGGCGTGGCGCGGGGCTCGCCGTGCCGGGCGGGGGG
TGGCGGCAGGTGGGGGTGCCGGGCGGGGCGGGGCCGCCTCGGGCCGGGGAGGGCT
CGGGGGAGGGGCGCGGCGGCCCCCGGAGCGCCGGCGGCTGTCGAGGCGCGGCGAG
CCGCAGCCATTGCCTTTTATGGTAATCGTGCGAGAGGGCGCAGGGACTTCCTTTGTC
CCAAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGCCTTCG
TGCGTCGCCGCGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCTGGCGTGTGA
CCGGCGGCTCTAGAGCCTCTGCTAACCATGTTCATGCCTTCTTCTTTTTCCTACAGCT
CCTGGGCAACGTGCTGGTTATTGTGCTGTCTCATCATTTTGGCAAAGAATTCGATAT
CAAGCTTGCTAGCGCCACCATGACTTCCAGTCCAAAGGCGCCTCCTGGAGAGCAAG
GACGCAAGGAAGCAGAGATGCCCGGAACCGCGCTGTCTCCACTGCTCTTGTTGCTG
CTCCTGTCCTGGGCGTCCCGGAACGAAGCAGCTCCGGACCAGGATGAAATCGATTG
TCTCCCCGGCCTGGCCAAGCAGCCCTCTTTCCGGCAATACTCCGGCTACCTCAGAGC
ATCGGACTCCAAGCACTTCCACTACTGGTTTGTGGAGTCGCAGAACGACCCAAAGA
ACAGCCCCGTGGTGCTTTGGCTTAACGGGGGTCCCGGCTGCAGCTCGCTCGATGGG
CTGCTTACAGAGCACGGCCCCTTTCTGATCCAGCCAGATGGTGTCACCCTGGAGTAC
AACCCCTATGCTTGGAACCTGATTGCCAACGTGCTGTATATCGAGTCCCCAGCTGGG
GTGGGCTTCTCCTACTCGGATGACAAGATGTACGTGACCAATGACACAGAGGTGGC
GGAGAACAATTATGAAGCCCTTAAAGACTTCTTCCGCCTCTTTCCGGAATACAAGG
ACAACAAACTTTTCCTGACAGGAGAGAGCTATGCTGGCATCTACATCCCCACCTTGG
CTGTACTGGTCATGCAGGATCCTAGCATGAATCTTCAGGGGCTGGCTGTGGGCAAT
GGACTTGCCTCCTATGAGCAGAACGACAACTCCCTGGTCTACTTTGCCTACTACCAT
GGCCTTCTGGGGAACAGACTTTGGACTTCACTGCAGACCCACTGCTGCGCTCAGAA
CAAGTGTAACTTCTATGACAACAAAGACCCAGAGTGTGTAAACAATCTCCTGGAAG
TGTCTCGAATTGTGGGCAAATCTGGCCTCAACATCTACAATCTCTATGCTCCGTGTG
CTGGTGGGGTGCCCGGCAGACATAGATATGAGGACACACTTGTAGTCCAGGATTTT
GGCAACATCTTCACTCGCCTGCCACTTAAGCGGAGATTTCCTGAGGCACTGATGCGT
TCTGGGGACAAGGTACGCTTGGATCCTCCCTGCACCAACACCACAGCCCCTTCCAAC
TACCTCAACAACCCCTATGTTCGGAAGGCTCTCCACATCCCCGAGTCGCTGCCCCGC
TGGGACATGTGCAACTTCTTGGTGAATTTACAGTACCGCCGCCTCTACCAAAGCATG
AACTCCCAGTACCTGAAGCTGCTCAGTTCACAGAAATACCAGATCCTGCTCTACAAC
GGAGATGTGGACATGGCCTGCAACTTCATGGGCGATGAGTGGTTTGTGGATTCGCT
CAACCAGAAGATGGAGGTGCAGCGCCGGCCCTGGCTAGTGGACTACGGGGAGAGC
GGAGAACAGGTAGCTGGTTTCGTGAAGGAGTGTTCACACATCACCTTCCTCACCATC
AAGGGTGCCGGACACATGGTCCCCACGGACAAGCCTCGAGCTGCTTTTACCATGTT
CTCGAGGTTCCTGAACAAAGAGCCTTACTGACTCGAGCGGCCGCTTCGAGCAGACA
TGATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTGCA
ATAAACAAGTTAACAACAACAATTGCATTCATTTTATGTTTCAGGTTCAGGGGGAG
ATGTGGGAGGTTTTTTAAAGCAAGTAAAACCTCTACAAATGTGGTAAAATCGATAA
GGATCTGAGGAACCCCTAGTGATGGAGTTGGCCACTCCCTCTCTGCGCGCTCGCTCG
CTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACCCCCCCCCCCCCCCCC
CTGCAGCCTGGCGTAATAGCGAAGAGGCCCGCACCGATCGCCCTTCCCAACAGTTG
CGTAGCCTGAATGGCGAATGGCGCGACGCGCCCTGTAGCGGCGCATTAAGCGCGGC
GGGTGTGGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCC
AAAAAACTTGATTAGGGTGATGGTTCACGTAGTGGGCCATCGCCCTGATAGACGGT
TTTTCGCCCTTTGACGTTGGAGTCCACGTTCTTTAATAGTGGACTCTTGTTCCAAACT
GGAACAACACTCAACCCTATCTCGGTCTATTCTTTTGATTTATAAGGGATTTTGCCG
ATTTCGGCCTATTGGTTAAAAAATGAGCTGATTTAACAAAAATTTAACGCGAATTTT
AACAAAATATTAACGTTTACAATTTCCTGATGCGGTATTTTCTCCTTACGCATCTGTG
CGGTATTTCACACCGCATATGGTGCACTCTCAGTACAATCTGCTCTGATGCCGCATA
GTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTC
AGAGGTTTTCACCGTCATCACCGAAACGCGCGAGACGAAAGGGCCTCGTGATACGC
CTATTTTTATAGGTTAATGTCATGATAATAATGGTTTCTTAGACGTCAGGTGGCACT
TTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
ATGTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCTTA
CGGATGGCATGACAGTAAGAGAATTATGCAGTGCTGCCATAACCATGAGTGATAAC
ACTGCGGCCAACTTACTTCTGACAACGATCGGAGGACCGAAGGAGCTAACCGCTTT
TTTGCACAACATGGGGGATCATGTAACTCGCCTTGATCGTTGGGAACCGGAGCTGA
ATGAAGCCATACCAAACGACGAGCGTGACACCACGATGCCTGTAGCAATGGCAACA
ACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTGCC
TCACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTA
GAAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTACC
AACTCTTTTTCCGAAGGTAACTGGCTTCAGCAGAGCGCAGATACCAAATACTGTCCT
TCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGAGA
TACCTACAGCGTGAGCATTGAGAAAGCGCCACGCTTCCCGAAGGGAGAAAGGCGG
ACAGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAA
GAGCGCCCAATACGCAAACCGCCTCTCCCCGCGCGTTGGCCGATTCATTAATGCAG
GGCTGCAGGGGGGGGGGGGGGGG

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method comprising administering to a cell:
(i) a first isolated nucleic acid comprising a transgene engineered to express a β-galactosidase (GLB) protein; and
(ii) a second isolated nucleic acid comprising a transgene engineered to express a cathepsin protein.

2. The method of claim 1, wherein the β-galactosidase protein is a human GLB protein (e.g., hGLB1) or a mouse GLB protein (e.g., mGLB1).

3. The method of claim 1 or 2, wherein the cathepsin protein is a cathepsin A protein (CTSA).

4. The method of claim 3, wherein the cathepsin A protein is human cathepsin A (hCTSA) or mouse cathepsin A (mCTSA).

5. The method of any one of claims 1 to 4, wherein the GLB protein comprises or consists of the sequence set forth in SEQ ID NO: 5 or 6.

6. The method of any one of claims 1 to 5, wherein the CTSA protein comprises or consists of the sequence set forth in SEQ ID NO:7 or 8.

7. The method of any one of claims 1 to 6, wherein the first isolated nucleic acid comprises adeno-associated virus (AAV) inverted terminal repeats (ITRs) flanking the transgene.

8. The method of any one of claims 1 to 7, wherein the second isolated nucleic acid comprises adeno-associated virus (AAV) inverted terminal repeats (ITRs) flanking the transgene.

9. The method of claim 7 or 8, wherein at least one of the ITRs flanking the transgene of the first isolated nucleic acid is a mutant ITR (mTR) or a ΔITR.

10. The method of claim 8 or 9, wherein at least one of the ITRs flanking the transgene of the second isolated nucleic acid is a mutant ITR (mTR) or a ΔITR.

11. The method of any one of claims 1 to 10, wherein the transgene of the first isolated nucleic acid, the transgene of the second isolated nucleic acid, or the transgene of the first isolated nucleic acid and the second isolated nucleic acid further comprise(s) a promoter, optionally wherein the promoter is CAG promoter.

12. The method of any one of claims 1 to 11, wherein the first isolated nucleic acid is located on a plasmid.

13. The method of any one of claims 1 to 12, wherein the second isolated nucleic acid is located on a plasmid.

14. The method of any one of claims 1 to 13, wherein the first isolated nucleic acid and the second isolated nucleic acid are located on the same plasmid.

15. The method of any one of claims 1 to 11, wherein the first isolated nucleic acid is encapsidated by an AAV capsid protein, optionally wherein the capsid protein is an AAV9 capsid protein.

16. The method of any one of claims 1 to 11 or claim 15, wherein the second isolated nucleic acid is encapsidated by an AAV capsid protein, optionally wherein the capsid protein is an AAV9 capsid protein.

17. The method of claim 15 or 16, wherein the first isolated nucleic acid and the second isolated nucleic acid are encapsidated together in an rAAV, optionally wherein the AAV comprises an AAV9 capsid protein.

18. The method of any one of claims 1 to 17, wherein the first isolated nucleic acid and/or the second isolated nucleic acid are administered by injection.

19. The method of any one of claims 1 to 18, wherein the first isolated nucleic acid and the second isolated nucleic acid are administered separately.

20. The method of any one of claims 1 to 18, wherein the first isolated nucleic acid and the second isolated nucleic acid are administered together (e.g., as part of the same composition).

21. The method of any one of claims 1 to 20, wherein the first isolated nucleic acid and the second isolated nucleic acid are administered simultaneously (e.g., at the same time).

22. The method of any one of claims 1 to 20, wherein the cell is in vivo.

23. The method of claim 22, wherein the cell is in a subject, optionally wherein the subject is a human.

24. An isolated nucleic acid comprising the sequence set forth in any one of SEQ ID NOs: 1-4.

25. An isolated nucleic acid comprising the sequence set forth in any one of SEQ ID NO: 9-12.

26. A composition comprising:
(i) a first recombinant adeno-associated virus (rAAV) comprising a transgene engineered to express a β-galactosidase (GLB) protein; and
(ii) a second rAAV, comprising a transgene engineered to express a cathepsin protein.

27. The composition of claim 26, further comprising a pharmaceutically acceptable excipient.

28. The composition of claim 26 or 27, wherein the first rAAV and/or the second rAAV comprises an AAV9 capsid protein.

29. The composition of any one of claims 26 to 28, wherein the β-galactosidase protein is a human GLB protein (e.g., hGLB1) or a mouse GLB protein (e.g., mGLB1).

30. The composition of any one of claims 26 to 29, the cathepsin protein is a cathepsin A protein (CTSA), optionally wherein the cathepsin A protein is human cathepsin A (hCTSA) or mouse cathepsin A (mCTSA).

31. The composition of any one of claims 26 to 30, wherein the GLB protein comprises or consists of the sequence set forth in SEQ ID NO: 5 or 6.

32. The method of any one of claims 26 to 31, wherein the CTSA protein comprises or consists of the sequence set forth in SEQ ID NO: 7 or 8.

33. The composition of any one of claims 26 to 32, wherein the ratio of the first rAAV to the second rAAV is 0.5:1, 1:1, 1:2, 1:4, 1:10, 1:0.5, 2:1, 4:1, or 10:1.

34. The composition of any one of claims 26 to 32, wherein the ratio of the second rAAV to the first rAAV is 0.5:1, 1:1, 1:2, 1:4, 1:10, 1:0.5, 2:1, 4:1, or 10:1.

35. The composition of any one of claims 26 to 33, wherein the composition is formulated for injection.

36. A recombinant adeno-associated virus (rAAV) comprising a transgene engineered to express:
(i) a β-galactosidase (GLB) protein, or a portion thereof; and
(ii) a cathepsin protein or a portion thereof.

37. The rAAV of claim 36, wherein the β-galactosidase protein is a human GLB protein (e.g., hGLB1) or a mouse GLB protein (e.g., mGLB1).

38. The rAAV of claim 36 or 37, wherein the cathepsin protein is a cathepsin A protein (CTSA), optionally wherein the cathepsin A protein is human cathepsin A (hCTSA) or mouse cathepsin A (mCTSA).

39. The rAAV of any one of claims 36 to 38, wherein the GLB protein comprises or consists of the sequence set forth in SEQ ID NO: 5 or 6.

40. The rAAV of any one of claims 36 to 39, wherein the CTSA protein comprises or consists of the sequence set forth in SEQ ID NO: 7 or 8.

41. A method for treating a lysosomal storage disease, the method comprising administering to a subject having a lysosomal storage disease:
(i) a first isolated nucleic acid comprising a transgene engineered to express a β-galactosidase (GLB) protein; and
(ii) a second isolated nucleic acid comprising a transgene engineered to express a cathepsin protein.

42. The method of claim 41, wherein the β-galactosidase protein is a human GLB protein (e.g., hGLB1) or a mouse GLB protein (e.g., mGLB1).

43. The method of claim 41 or 42, wherein the cathepsin protein is a cathepsin A protein (CTSA).

44. The method of claim 43, wherein the cathepsin A protein is human cathepsin A (hCTSA) or mouse cathepsin A (mCTSA).

45. The method of any one of claims 41 to 44, wherein the GLB protein comprises or consists of the sequence set forth in SEQ ID NO: 5 or 6.

46. The method of any one of claims 41 to 45, wherein the CTSA protein comprises or consists of the sequence set forth in SEQ ID NO: 7 or 8.

47. The method of any one of claims 41 to 46, wherein the first isolated nucleic acid comprises adeno-associated virus (AAV) inverted terminal repeats (ITRs) flanking the transgene.

48. The method of any one of claims 41 to 47, wherein the second isolated nucleic acid comprises adeno-associated virus (AAV) inverted terminal repeats (ITRs) flanking the transgene.

49. The method of claim 47 or 48, wherein at least one of the ITRs flanking the transgene of the first isolated nucleic acid is a mutant ITR (mTR) or a ΔITR.

50. The method of claim 48 or 49, wherein at least one of the ITRs flanking the transgene of the second isolated nucleic acid is a mutant ITR (mTR) or a ΔITR.

51. The method of any one of claims 41 to 50, wherein the transgene of the first isolated nucleic acid, the transgene of the second isolated nucleic acid, or the transgene of the first isolated nucleic acid and the second isolated nucleic acid further comprise(s) a promoter, optionally wherein the promoter is CAG promoter.

52. The method of any one of claims 41 to 51, wherein the first isolated nucleic acid is encapsidated by an AAV capsid protein, optionally wherein the capsid protein is an AAV 9 capsid protein.

53. The method of any one of claims 41 to 52, wherein the second isolated nucleic acid is encapsidated by an AAV capsid protein, optionally wherein the capsid protein is an AAV9 capsid protein.

54. The method of any one of claims 41 to 52, wherein the first isolated nucleic acid and the second isolated nucleic acid are encapsidated together in an rAAV, optionally wherein the AAV comprises an AAV9 capsid protein.

55. A method for treating a lysosomal storage disease, the method comprising administering to a subject having a lysosomal storage disease the composition of any one of claims 26 to 35.

56. A method for treating a lysosomal storage disease, the method comprising administering to a subject having a lysosomal storage disease the rAAV of any one of claims 36 to 39.

57. The method of any one of claims 41 to 56, wherein the lysosomal storage disease is GM1-gangliosidosis.


SEQUENCE LISTING (12 sequences)

SEQ ID NO: 1 - DNA, 2034 bp, Artificial Sequence
  Synthetic Polynucleotide
atgccggggttcctggttcgcatcctccttctgctgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatga

SEQ ID NO: 2 - DNA, 1944 bp, Artificial Sequence
  Synthetic Polynucleotide
atgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacgggggaaggctggagccacgctggacatcctggtggagaacatggggcgtgtgaactatggcagattcatcaatgacttcaagggtttgatttccaacatgactatcaactccactgtcctcaccaactggacggtcttcccactgaacactgaggccatggtacgcaaccatctctggggccgggaggccagtgatgagggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctga

SEQ ID NO: 3 - DNA, 1497 bp, Artificial Sequence
  Synthetic Polynucleotide
atgacttccagtccccgggcgcctcctggagagcaaggacgcgggggagcagagatgatccgagcagcgcctccaccactgttcttgttgctgctcctgttgctgttgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactga

SEQ ID NO: 4 - DNA, 1479 bp, Artificial Sequence
  Synthetic Polynucleotide
atgacttccagtccaaaggcgcctcctggagagcaaggacgcaaggaagcagagatgcccggaaccgcgctgtctccactgctcttgttgctgctcctgtcctgggcgtcccggaacgaagcagctccggaccaggatgaaatcgattgtctccccggcctggccaagcagccctctttccggcaatactccggctacctcagagcatcggactccaagcacttccactactggtttgtggagtcgcagaacgacccaaagaacagccccgtggtgctttggcttaacgggggtcccggctgcagctcgctcgatgggctgcttacagagcacggcccctttctgatccagccagatggtgtcaccctggagtacaacccctatgcttggaacctgattgccaacgtgctgtatatcgagtccccagctggggtgggcttctcctactcggatgacaagatgtacgtgaccaatgacacagaggtggcggagaacaattatgaagcccttaaagacttcttccgcctctttccggaatacaaggacaacaaacttttcctgacaggagagagctatgctggcatctacatccccaccttggctgtactggtcatgcaggatcctagcatgaatcttcaggggctggctgtgggcaatggacttgcctcctatgagcagaacgacaactccctggtctactttgcctactaccatggccttctggggaacagactttggacttcactgcagacccactgctgcgctcagaacaagtgtaacttctatgacaacaaagacccagagtgtgtaaacaatctcctggaagtgtctcgaattgtgggcaaatctggcctcaacatctacaatctctatgctccgtgtgctggtggggtgcccggcagacatagatatgaggacacacttgtagtccaggattttggcaacatcttcactcgcctgccacttaagcggagatttcctgaggcactgatgcgttctggggacaaggtacgcttggatcctccctgcaccaacaccacagccccttccaactacctcaacaacccctatgttcggaaggctctccacatccccgagtcgctgccccgctgggacatgtgcaacttcttggtgaatttacagtaccgccgcctctaccaaagcatgaactcccagtacctgaagctgctcagttcacagaaataccagatcctgctctacaacggagatgtggacatggcctgcaacttcatgggcgatgagtggtttgtggattcgctcaaccagaagatggaggtgcagcgccggccctggctagtggactacggggagagcggagaacaggtagctggtttcgtgaaggagtgttcacacatcaccttcctcaccatcaagggtgccggacacatggtccccacggacaagcctcgagctgcttttaccatgttctcgaggttcctgaacaaagagccttactga

SEQ ID NO: 5 - protein, 677 aa, Homo sapiens
MPGFLVRILPLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 6 - protein, 647 aa, Mus musculus
MLRVPLCTPLPLLALLQLLGAAHGIYNVTQRTFKLDYSRDRFLKDGQPFRYISGSIHYFRIPRFYWEDRLLKMKMAGLNAIQMYVPWNFHEPQPGQYEFSGDRDVEHFIQLAHELGLLVILRPGPYICAEWDMGGLPAWLLEKQSIVLRSSDPDYLVAVDKWLAVLLPKMKPLLYQNGGPIITVQVENEYGSYFACDYDYLRFLVHRFRYHLGNDVILFTTDGASEKMLKCGTLQDLYATVDFGTGNNITQAFLVQRKFEPKGPLINSEFYTGWLDHWGKPHSTVKTKTLATSLYNLLARGANVNLYMFIGGTNFAYWNGANTPYEPQPTSYDYDAPLSEAGDLTKKYFALREVIQMFKEVPEGPIPPSTPKFAYGKVALRKFKTVAEALGILCPNGPVKSLYPLTFTQVKQYFGYVLYRTTLPQDCSNPKPIFSSPFNGVRDRAYVSVDGVPQGILDRNLMTALNIRGKAGATLDILVENMGRVNYGRFINDFKGLISNMTINSTVLTNWTVFPLNTEAMVRNHLWGREASDEGHLDGRSTSNSSDLILPTFYVGNFSIPSGIPDLPQDTFIQFPGWSKGQVWINGFNLGRYWPTMGPQKTLFVPRNILTTSAPNNITVLELEFAPCSEGTPELCTVEFVDTPVIS

SEQ ID NO: 7 - protein, 498 aa, Homo sapiens
MTSSPRAPPGEQGRGGAEMIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 8 - protein, 492 aa, Mus musculus
MTSSPKAPPGEQGRKEAEMPGTALSPLLLLLLLSWASRNEAAPDQDEIDCLPGLAKQPSFRQYSGYLRASDSKHFHYWFVESQNDPKNSPVVLWLNGGPGCSSLDGLLTEHGPFLIQPDGVTLEYNPYAWNLIANVLYIESPAGVGFSYSDDKMYVTNDTEVAENNYEALKDFFRLFPEYKDNKLFLTGESYAGIYIPTLAVLVMQDPSMNLQGLAVGNGLASYEQNDNSLVYFAYYHGLLGNRLWTSLQTHCCAQNKCNFYDNKDPECVNNLLEVSRIVGKSGLNIYNLYAPCAGGVPGRHRYEDTLVVQDFGNIFTRLPLKRRFPEALMRSGDKVRLDPPCTNTTAPSNYLNNPYVRKALHIPESLPRWDMCNFLVNLQYRRLYQSMNSQYLKLLSSQKYQILLYNGDVDMACNFMGDEWFVDSLNQKMEVQRRPWLVDYGESGEQVAGFVKECSHITFLTIKGAGHMVPTDKPRAAFTMFSRFLNKEPY

SEQ ID NO: 9 - DNA, 7071 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgccggggttcctggttcgcatcctccttctgctgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgactcgagtttttttttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 10 - DNA, 6980 bp, Artificial Sequence
  Synthetic Polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcccaccatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacgggggaaggctggagccacgctggacatcctggtggagaacatggggcgtgtgaactatggcagattcatcaatgacttcaagggtttgatttccaacatgactatcaactccactgtcctcaccaactggacggtcttcccactgaacactgaggccatggtacgcaaccatctctggggccgggaggccagtgatgagggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgactcgagtttttttttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 11 - DNA, 6813 bp, Artificial Sequence
  Synthetic Polynucleotide
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgacttccagtccccgggcgcctcctggagagcaaggacgcgggggagcagagatgatccgagcagcgcctccaccactgttcttgttgctgctcctgttgctgttgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgactcgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagggggggggggggggg

SEQ ID NO: 12 - DNA, 6797 bp, Artificial Sequence
  Synthetic Polynucleotide
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgacttccagtccaaaggcgcctcctggagagcaaggacgcaaggaagcagagatgcccggaaccgcgctgtctccactgctcttgttgctgctcctgtcctgggcgtcccggaacgaagcagctccggaccaggatgaaatcgattgtctccccggcctggccaagcagccctctttccggcaatactccggctacctcagagcatcggactccaagcacttccactactggtttgtggagtcgcagaacgacccaaagaacagccccgtggtgctttggcttaacgggggtcccggctgcagctcgctcgatgggctgcttacagagcacggcccctttctgatccagccagatggtgtcaccctggagtacaacccctatgcttggaacctgattgccaacgtgctgtatatcgagtccccagctggggtgggcttctcctactcggatgacaagatgtacgtgaccaatgacacagaggtggcggagaacaattatgaagcccttaaagacttcttccgcctctttccggaatacaaggacaacaaacttttcctgacaggagagagctatgctggcatctacatccccaccttggctgtactggtcatgcaggatcctagcatgaatcttcaggggctggctgtgggcaatggacttgcctcctatgagcagaacgacaactccctggtctactttgcctactaccatggccttctggggaacagactttggacttcactgcagacccactgctgcgctcagaacaagtgtaacttctatgacaacaaagacccagagtgtgtaaacaatctcctggaagtgtctcgaattgtgggcaaatctggcctcaacatctacaatctctatgctccgtgtgctggtggggtgcccggcagacatagatatgaggacacacttgtagtccaggattttggcaacatcttcactcgcctgccacttaagcggagatttcctgaggcactgatgcgttctggggacaaggtacgcttggatcctccctgcaccaacaccacagccccttccaactacctcaacaacccctatgttcggaaggctctccacatccccgagtcgctgccccgctgggacatgtgcaacttcttggtgaatttacagtaccgccgcctctaccaaagcatgaactcccagtacctgaagctgctcagttcacagaaataccagatcctgctctacaacggagatgtggacatggcctgcaacttcatgggcgatgagtggtttgtggattcgctcaaccagaagatggaggtgcagcgccggccctggctagtggactacggggagagcggagaacaggtagctggtttcgtgaaggagtgttcacacatcaccttcctcaccatcaagggtgccggacacatggtccccacggacaagcctcgagctgcttttaccatgttctcgaggttcctgaacaaagagccttactgactcgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcagggggggggggggggg